SEQUENCE LISTING (1 sequence)

SEQ ID NO: 1 - DNA, 175 bp, synthetic construct
  misc_feature 1..175: note = Synthetic DNA (PCR product)
  source 1..175: mol_type = other DNA
catcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcaca

(19) United States
(12) Patent Application Publication
(10) Pub. No.: US 2024/0352538 A1
(43) Pub. Date: Oct. 24, 2024

(54) METHODS AND KITS FOR ANALYZING NUCLEOSOMES AND PLASMA PROTEINS

(71) Applicants: Yeda Research and Development Co. Ltd., Rehovot (IL); SeqLL Inc., Billerica, MA (US)

(72) Inventors: Efrat SHEMA-YAACOBY, Moshav Sitriya (IL); Erez NIR, Rehovot (IL); Vadim FEDYUK, Rehovot (IL); Ekaterina ANDREISHCHEVA, Ipswich, MA (US); Daniel JONES, Boston, MA (US); Abhijeet SHINDE, Sudbury, MA (US)

(73) Assignees: Yeda Research and Development Co. Ltd., Rehovot (IL); SeqLL Inc., Billerica, MA (US)

(21) Appl. No.: 18/764,250

(22) Filed: Jul. 4, 2024

Related U.S. Application Data
(63) Continuation of application No. PCT/IL2022/051160, filed on Nov. 3, 2022.
(60) Provisional application No. 63/296,505, filed on Jan. 5, 2022.

(51) Int. Cl. C12Q 1/6886; C12Q 1/6804

(52) U.S. Cl. CPC C12Q 1/6886; C12Q 1/6804; C12Q 2600/156

(57) ABSTRACT

A method of analyzing nucleosomes is provided. The method comprising: (a) isolating a plurality of nucleosome molecules from a biological sample; (b) enzymatically linking adenine nucleotides to free DNA ends of the plurality of nucleosome molecules, wherein at least a portion of the adenine nucleotides comprises a label, such that the plurality of nucleosome molecules become attached to a labeled poly(A) tail; (c) hybridizing the plurality of nucleosome molecules attached to the labeled poly(A) tail to a solid support coated with poly(T); and (d) imaging the solid support, whereby the plurality of nucleosome molecules are visualized.

### RELATED APPLICATIONS

This application is a Continuation of PCT Patent Application No. PCT/IL2022/051160 having International filing date Nov. 3, 2022, which claims the benefit of priority under 35 USC § 119(e) of U.S. Provisional Patent Application No. 63/296,505 filed on Jan. 5, 2022. The contents of the above applications are all incorporated by reference as if fully set forth herein in their entirety.

### SEQUENCE LISTING STATEMENT

The XML file, entitled 100603Sequence Listing.xml, created on Jun. 5, 2024, comprising 2,323 bytes, submitted concurrently with the filing of this application is incorporated herein by reference.

### FIELD AND BACKGROUND OF THE INVENTION

The present invention, in some embodiments thereof, relates to methods and kits for analyzing nucleosomes and plasma proteins.

Late stage cancers often lack an effective treatment option. Survival rates increase significantly when cancer is identified at early stages, as the tumor can be surgically removed or treated with milder drug regimens; average 5-year survival at early stage is 91%, while average 5-year survival at late stage is 26%. Detection of tumors at the earliest possible stage is therefore of paramount importance for cancer treatment. Currently, a limited number of screening tests exist for a few cancer types, including colonoscopy, prostate specific antigen, mammography, and cervical cytology. However, the efficacy of some tests has been questioned, and many patients do not follow medical guidelines for screening. Most cancer types currently lack an effective non-invasive early screening option.

Non-invasive liquid biopsy methods, based on the analysis of cfDNA, potentiate a new generation of diagnostic approaches. The cfDNA that circulates in the plasma and serum of healthy individuals originates predominantly from death of normal blood cells (1). In cancer patients, however, a fraction of cfDNA is tumor-derived, termed circulating tumor DNA (ctDNA). ctDNA-based sequence analysis has been shown to reveal tumor-specific genetic alterations and provide the means for non-invasive molecular profiling of tumors (2, 3). Despite encouraging data, these approaches are limited, as they require genetic differences (i.e. mutations) in order to distinguish between the normal and tumor DNA. Liquid biopsy approaches based on analysis of non-genetic features have emerged recently, most prominently methodologies that utilize tissue- and cancer-specific DNA methylation, as well as differential fragmentation patterns of cfDNA (4 8). cfDNA in the plasma appears predominantly in the form of nucleosomes (cfNucleosomes), the basic unit of chromatin that consists of ˜150 base pairs of DNA wrapped around the octamer of core histone proteins. Histones are extensively modified by covalent attachment of various chemical groups, forming combinatorial epigenetic patterns that are unique to each tissue, and provide information on gene expression and regulatory elements within cells (9 12). There is evidence that cfNucleosomes retain at least some of their epigenetic modifications, and a recent study applied Chromatin Immunoprecipitation and sequencing (ChIP-seq) to identify certain marks (13 15). Moreover, deep sequencing of cfDNA revealed nucleosome occupancy patterns correlating with the tissue of origin (16 18). While these approaches provide the first glimpse into the rich epigenetic information present in plasma that has so far remained mostly inaccessible, they have major limitations. Mainly, they require large amounts of input material, have a limited dynamic range (ChIP-seq), or are costly and require deep sequencing. Most importantly, these methodologies have limited output and sensitivity, as they usually measure a single layer of information (i.e. DNA methylation or a single histone modification or nucleosome occupancy, etc.). Thus, high-resolution approaches that integrate information from multiple parameters spanning different types of analytes are required.

Additional background art includes:
WO2017034970
Fedyuk et al. DOI www(dot)doi(dot)org/10.1101/2021.11.01.466724
U.S. Pat. Publ. No. 20190308190
Mao et al. Sci Adv. 2021 August; 7(33): eabg6522

### SUMMARY OF THE INVENTION

According to an aspect of some embodiments of the present invention there is provided a method of analyzing nucleosomes, the method comprising:
(a) isolating a plurality of nucleosome molecules from a biological sample;
(b) enzymatically linking adenine nucleotides to free DNA ends of the plurality of nucleosome molecules, wherein at least a portion of the adenine nucleotides comprises a label, such that the plurality of nucleosome molecules become attached to a labeled poly(A) tail;
(c) hybridizing the plurality of nucleosome molecules attached to the labeled poly(A) tail to a solid support coated with poly(T); and
(d) imaging the solid support, whereby the plurality of nucleosome molecules are visualized.

According to some embodiments of the invention, the method further comprises
(e) incubating the solid support with at least one labeling ligand with specific binding affinity for a target molecule of the nucleosome and wherein the labeling ligand includes a marker;
(f) imaging the solid support, whereby the plurality of nucleosome molecules comprising the target molecule are visualized.

According to some embodiments of the invention, the enzymatically linking comprises using a template-dependent DNA polymerase and a Terminal deoxynucleotidyl transferase (TdT).

According to some embodiments of the invention, the template-dependent DNA polymerase comprises a Klenow polymerase.

According to some embodiments of the invention, the biological sample comprises a biological fluid.

According to some embodiments of the invention, the biological fluid is selected from the group consisting of a plasma, a serum, a blood, urine, saliva, a lymph fluid and a synovial fluid.

According to some embodiments of the invention, the biological fluid is plasma.

According to some embodiments of the invention, a volume of the plasma is less than 1 ml.

According to some embodiments of the invention, the method further comprises cleaving the label and optionally washing it prior to step (e).

According to some embodiments of the invention, the label comprises a fluorophore.

According to some embodiments of the invention, the fluorophore is selected from the group consisting of Alexa 488, Alexa 555, Alexa 640, CY3, CY5, an Atto Dyes and a Pacific Dye.

According to some embodiments of the invention, the labeling ligand comprises an antibody.

According to some embodiments of the invention, the labeling ligand comprises a fluorophore.

According to some embodiments of the invention, the labeling ligand comprises Alexa fluor.

According to some embodiments of the invention, the target molecule is a post translational modification.

According to some embodiments of the invention, the target molecule is a histone modification and/or a histone variant.

According to some embodiments of the invention, the histone variant is selected from the group consisting of macroH2A1.1, macroH2A1.2, H2AZ, H2AX, H3.1 and H3.3.

According to some embodiments of the invention, the histone modification is selected from the group consisting of acetylation, methylation, phosphorylation, ribosylation, citrullination, ubiquitination, hydroxylation, glycosylation, nitrosylation, glutamination and isomerisation.

According to some embodiments of the invention, the histone modification is selected from the group consisting of H2B Ser 14 (Phos), H3 Ser 10 (Phos), H3 Lys 9 (Me), H3 Lys 27 (Me), H3 Lys 36 (Me), H3 Lys 79 (Me), H4 Lys 20 (Me), H3 Lys 4 (Me), H3 Lys 9 (Ac), H3 Lys 14 (Ac), H3 Lys 23 (Ac), H4 arg 3 (Me), H3 Lys 27 (Ac), H4 arg 3 (Me), H4 lys 5 (Ac), H4 Ser 2 (phos), H4 Arg 3(me), H4 Lys 5 (Ac) and H3 Lys 18 (Ac).

According to some embodiments of the invention, the target molecule is a nucleotide modification.

According to some embodiments of the invention, the nucleotide modification is selected from the group consisting of 5-methyl-(5-mC), 5-hydroxymethyl-(5-hmC), 5-formyl-(5-fC) and 5-carboxy-(5-eaC) cytosine.

According to some embodiments of the invention, the imaging of step (f) comprises time lapse imaging.

According to some embodiments of the invention, the imaging of step (f) and optionally (d) comprises TIRF microscopy.

According to some embodiments of the invention, the target molecule comprises a plurality of target molecules.

According to some embodiments of the invention, the method further comprises repeating steps (e) and (f) with additional labeling ligna distinctive of the labeling ligand such as in binding a different target molecule of the nucleosome.

According to some embodiments of the invention, the imaging of step (e) comprises multiplex imaging.

According to some embodiments of the invention, the plurality of nucleosome molecules comprise cell-free nucleosomes (cfNucleosomes).

According to some embodiments of the invention, the solid support is coated with poly ethylene glycol (PEG).

According to some embodiments of the invention, the method further comprises sequencing DNA of the plurality of nucleosome molecules.

According to some embodiments of the invention, the sequencing comprises sequencing by synthesis.

According to an aspect of some embodiments of the present invention there is provided a method of diagnosing a disease associated with modified, cell-free nucleosomes (cfNucleosomes) comprising analyzing nucleosome molecules in a biological fluid according to the method as described herein, wherein presence of a pathological nucleorise phenotype is indicative of a disease associated with modified cfNucleosomes.

According to some embodiments of the invention, the phenotype is selected from the group consisting of:
(i) increased concentration of nucleosomes as compared to same in a non-cancerous biological fluid;
(ii) altered percentage of modified nucleosome in a specific target molecule in the biological fluid as compared to same in a non-cancerous biological fluid;
(iii) altered ratio between a plurality of the target molecules as compared to same in a non-cancerous biological fluid;
(iv) altered percentage of nucleosomes that comprise a combinatorial pattern of target molecules in a single nucleosome as compared to same in a biological fluid of a non-cancerous biological fluid.

According to some embodiments of the invention, the disease is colorectal cancer (CRC) and the phenotype is selected from the group consisting of:
(i) increased concentration of nucleosomes as compared to same in a non-cancerous biological fluid;
(ii) increased percentage of modified nucleosome in the biological fluid and wherein the modified nucleosome is comprises H3K27me3-, H3K9me3-, H3K9ac- and H3K4me1;
(iii) higher ratio between H3K9ac to H3K4me1 as compared to same in a non-cancerous biological fluid;
(iv) a decrease in nucleosomes having a bivalent pattern of H3K9me3+H3K36me3- and an increase in nucleosomes having a bivalent pattern of H3K4me3+H3K27me3 as compared to same in a non-cancerous biological fluid.

According to some embodiments of the invention, the disease is cancer.

According to some embodiments of the invention, the disease is colorectal cancer.

According to some embodiments of the invention, the disease is selected from the group consisting of pre-malignant and malignant neoplasms, histocytoma, glioma, astrocyoma, osteoma, lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma, leukemias, systemic lupus erythematosus, psoriasis, bone diseases, fibroproliferative disorders of connective tissue, cataracts and atherosclerosis.

According to some embodiments of the invention, the target molecule is a histone modification.

According to some embodiments of the invention, the histone modification comprises at least one of H3K9me3, H3K27me3, H3K4me3, H3K36me3, H3K9ac and H3K4me1.

According to some embodiments of the invention, the at least one comprises at least two.

According to some embodiments of the invention, the at least two comprise 2, 3, 4, 5 or 6.

According to an aspect of some embodiments of the present invention there is provided a method of treating a subject diagnosed with a disease associated with modified, cell-free nucleosomes (cfNucleosomes) in a subject, the method comprising:
(a) affirming diagnosis of the disease according to the method as described herein;
(b) administering a treatment to the disease.

According to an aspect of some embodiments of the present invention there is provided a method of identifying a tissue origin of a nucleosome molecule, the method comprising analyzing nucleosome according to the method as described herein, wherein abundance or pattern of the target molecule on the nucleosome is indicative of the tissue origin of the nucleosome.

According to an aspect of some embodiments of the present invention there is provided a kit comprising:
(a) a solid support;
(b) a Klenow polymerase; template dependent dna polymerase
(c) Terminal deoxynucleotidyl transferase (TdT);
(d) adenine nucleotides, wherein at least a portion of the adenine nucleotides comprises a label.

According to some embodiments of the invention, the kit further comprises at least one of;
(a) imaging buffer;
(b) at least one antibody to a target molecule on a nucleosome.

According to an aspect of some embodiments of the present invention there is provided a method of detecting at least one protein of interest, the method comprising:
(a) contacting a liquid biological sample with a solid support having immobilized thereto at least one capture antibody to the at least one protein of interest, wherein the contacting is under conditions which allow formation of immunocomplexes; and
(b) contacting the immunocomplexes with at least one labeled detection antibody to the at least one protein of interest, wherein the contacting is under conditions which allow formation of immunocomplexes between the immunocomplexes and the labeled detection antibody;
(c) imaging in a time-lapse manner the solid support using Total Internal Reflection (TIRF) microscopy such that the at least one protein of interest in the immunocomplexes is visualized via the labeled antibody, wherein the imaging is under non-flow conditions, thereby detecting the at least one protein of interest.

According to some embodiments of the invention, the at least one capture antibody is a polyclonal antibody.

According to some embodiments of the invention, the at least one capture antibody comprises a plurality of capture antibodies to distinct proteins of interest and wherein the at least one labeled detection antibody comprises a plurality of labeled detection antibodies to the comprising a plurality of distinct labels.

According to some embodiments of the invention, the solid support is PEG-avidin-coated solid support.

According to some embodiments of the invention, the capture antibody is biotinylated.

According to some embodiments of the invention, the imaging is effected in the presence of unbound labeled detection antibody to monitor association-dissociation events between the labeled detection antibody and the protein of interest.

According to some embodiments of the invention, the liquid biological sample comprises plasma.

According to some embodiments of the invention, the imaging in a time-lapse manner is effected for 1-24 hours.

According to some embodiments of the invention, the at least one protein of interest is a non-secreted tumor specific plasma protein.

According to some embodiments of the invention, the at least one protein of interest is a secreted tumor specific plasma protein.

According to some embodiments of the invention, the imaging is performed without prior washing of the at least one labeled detection antibody.

According to some embodiments of the invention, the labeled detection antibody comprises a fluorophore.

According to some embodiments of the invention, the biological fluid is selected from the group consisting of plasma, serum, blood, urine, saliva, lymph fluid and synovial fluid.

According to some embodiments of the invention, the biological fluid sample is undiluted and/or unprocessed.

According to some embodiments of the invention, the biological fluid sample is plasma and a volume of the plasma is less than 1 ml.

According to some embodiments of the invention, the at least one protein of interest is selected from the group consisting of a mutant oncoprotein, a mutant tumor suppressor protein and a pathogen-encoded oncoprotein derived from an oncogenic pathogen.

According to some embodiments of the invention, the at least one protein of interest is selected from the group consisting of p53, MST1, CEA, and TIMP-1.

According to some embodiments of the invention, the at least one protein of interest is selected from the group consisting of p53, TIMP-1, MST1, CEA, RAS, KRAS, BRAF, PIK3CA, EGFR, NOTCH1, P53, CDKN2A, PTEN, RB, APC, SMAD, ARID1A, MLL2, MLL3, GATA3, VHL and PBRM1.

According to an aspect of some embodiments of the present invention there is provided a method of diagnosing and optionally treating a disease associated with a protein of interest, the method comprising detecting the protein in a biological fluid sample of a subject in need thereof according to the method as described herein, wherein presence or level of the protein is indicative of the disease.

According to an aspect of some embodiments of the present invention there is provided a disease is cancer.

According to some embodiments of the invention, the disease is colorectal cancer.

According to some embodiments of the invention, the method further comprises selecting a treatment for the disease once it is diagnosed.

According to an aspect of some embodiments of the present invention there is provided a method of analyzing a liquid biological sample, the method comprising analyzing nucleosomes and a protein of interest according to the combined methods of nucleosome and protein detection as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-F show that EPINUC decodes the combinatorial epigenetic states of plasma cell-free nucleosomes. a, Experimental scheme: (1) Sample preparation procedure of cfNucleosomes is carried out in one-step and consists of two enzymatic processes: repair of DNA ends by Klenow polymerase, and addition of a poly A tail by Terminal Transferase (TdT). The reaction contains a mixture of natural dATPs and fluorescently labeled dATPs (Cy3-dATP) to label nucleosomes. (2) cfNucleosmes are captured on a PEGylated-poly T surface via dA:dT hybridization. Immobilized nucleosomes are incubated with fluorescently labeled antibodies targeting different histone modifications. (3) TIRF microscopy is applied to record nucleosome positions and generate time-lapse imaging of antibodies' binding events. b, Representative images of plasma-derived cfNucleosomes and the corresponding H3K4me3 and H3K27me3 signal (numbers within images represent counted spots). Each spot corresponds to a single nucleosome. Nucleosomes anchor to the surface specifically via hybridization, as evident from the lack of signal when tailed with dTTP (Poly-dT) rather than dATP (Poly-dA). c, Representative images of antibodies' binding and dissociation events over time from individual target molecules (marked by red/yellow circles). d,e,f, Example for quantification and representative images of the various parameters measured by EPINUC in plasma samples from a healthy subject and a CRC patient. Zoomed-in image segments of entire field of view (FOV, 148 μm²). Data is presented as the mean+/−standard deviation (s.d.) of 50 FOVs per sample. d, The percentage of cfNucleosomes (green spots, Cy3) that are modified by H3K27me3 (cyan spots, AF488). Red arrows indicate co-localization events. Scale bar=1 μm e, Ratio between H3K4me3 (red, AF647) and H3K27me3 (cyan) antibodies. White arrows indicate antibody spots, blue arrows indicate TetraSpeck beads that are used for alignment. Scale bar=5 μm. f, cfNucleosomes marked by the combinatorial pattern of both H3K27me3 and H3K4me3. Red arrows indicate co-localization events of H3K27me3 only, yellow arrow indicates a combinatorially modified nucleosome. Scale bar=1 μm.

FIGS. 2A-J show multiplexed single-molecule detection of cancer-associated protein biomarkers, mutant p53 and DNA methylation. a, Experimental scheme: biotinylated capture antibodies targeting distinct proteins are anchored to a PEG-streptavidin surface. Plasma proteins are captured on surface, followed by detection with fluorescently labeled antibodies and TIRF imaging. Multiplexed detection of up to three proteins is achieved by labeling antibodies with different fluorophores. Each spot represents a single protein bound on the surface. b, Representative TIRF images of selected CRC biomarkers measured simultaneously for each plasma sample: CEA (red), MST1 (cyan) and TIMP-1(green). Images reveal distinct biomarkers profiles for healthy and CRC. c, Representative TTRF images depicting α-CEA antibody binding events (spots) over serial plasma dilutions. d, Regression analysis of the number of spots as a function of plasma concentration highlights the linearity of detection. Data is presented as the mean+/−s.d. of 50 FOVs for each concentration. e,f,g Single-molecule detection of p53 in the plasma of healthy and CRC patients with known p53 mutations. e, Representative TTRF images. Detection is carried out simultaneously with antibodies targeting all p53 (red) and with antibodies that are specific to the mutant p53 conformation (green). Large diameter spots correspond to TetraSpeck beads used for alignment. f, p53 signal accumulation over time in late stage CRC and healthy plasma. Data is presented as the mean+/−s.d. of 50 FOVs for each time point. g, Total and mutant p53 levels in plasma show significantly higher levels in CRC patients versus healthy individuals (n=6 for each group). Box plots limits: 25-75% quantiles, middle: median, upper (lower) whisker to the largest (smallest) value no further than 1.5× interquartile range from the hinge. P values were calculated by Wilcoxon rank sum exact test. *** P value <0.001. h, Experimental scheme for single-molecule imaging of global DNA methylation: MBD2-biotin is incubated with Cy3-labeled (green) DNA, and binds specifically to methylated DNA molecules. Next, biotin-MBD-meDNA complexes are immobilized on a PEG-streptavidin surface, followed by TIRF imaging. Each spot represents a single bound complex, number of spots correspond to the level of DNA methylation in plasma. i, Representative TIRF images of DNA methylation in HEK293 cells treated with 5-Aza-2′-deoxycytidine, demonstrating significant reduction in methylation compared to control cells. j, Representative TIRF images of global cfDNA methylation levels in the plasma of CRC and healthy subjects, showing lower DNA methylation levels in CRC. For all images, numbers within images represent counted spots.

FIGS. 3A-H show that EPINUC reveals significant epigenetic and biomarkers alterations in the plasma of CRC patients. a, Representative TIRF images depict changes in protein biomarker levels in the plasma of healthy, CRC patient, and CRC following tumor resection. Numbers within images represent counted spots. b, CEA/MST1 normalized levels in the plasma of CRC patients and healthy individuals. Each bar represents a subject, data is presented as the mean+/−s.d. of 50 fields of view per sample. c, Box plot representation of the data in b (healthy=33, CRC=29). Box plots limits: 25-75% quantiles, middle: median, upper (lower) whisker to the largest (smallest) value no further than 1.5× interquartile range from the hinge. P values were calculated by Welch's t-test. *** P value <0.001. d, Histone PTMs, ratios and combinations (as indicated on the graphs) that significantly differ between healthy and CRC late stage samples (healthy=33, CRC=29). P values were calculated by Welch's t-test. * P value <0.05 ** P value <0.01. *** P value <0.001. e, Global DNA methylation levels, measured as in FIG. 2h, in the same cohort as (d). f, Histone PTMs, ratios and combinations (as indicated on the graphs) that significantly differ between healthy and early stage CRC patients (healthy=33, early CRC=8). P values were calculated by Wilcoxon rank sum exact test. * P value <0.05 ** P value <0.01. *** P value <0.001. g, Individual parameters predictive power score (PPS) analysis for the various subgroups (see Methods). Color scale represents PPS value. h, Principal Component Analysis (PCA) with input parameters of H3K27me3/Nuc, CEA/MST1 and CEA. Sample groups are color-coded as indicated, each dot represents a plasma sample.

FIGS. 4A-D show single-molecule imaging of MST1, TIMP-1 and mutant p53.
(a) Representative TIRF images (Left) and quantification (Right) of TIMP-1 protein levels in SW480 medium, following TIMP-1 knockdown versus control cells. Data is presented as the mean+/−s.d. of 50 FOVs for each treatment. (b, c) Representative TIRF images and standard curves of antibodies targeting MST1 and TIMP-1 on serial plasma dilutions, depicting linear detection of molecules within this concentration range. Data is presented as the mean+/−s.d. of 50 FOVs for each concentration. (d) Signal accumulation of mutant p53 over time for late stage CRC and healthy plasma samples. Each time point is presented as the mean+/−s.d. of 50 FOVs.

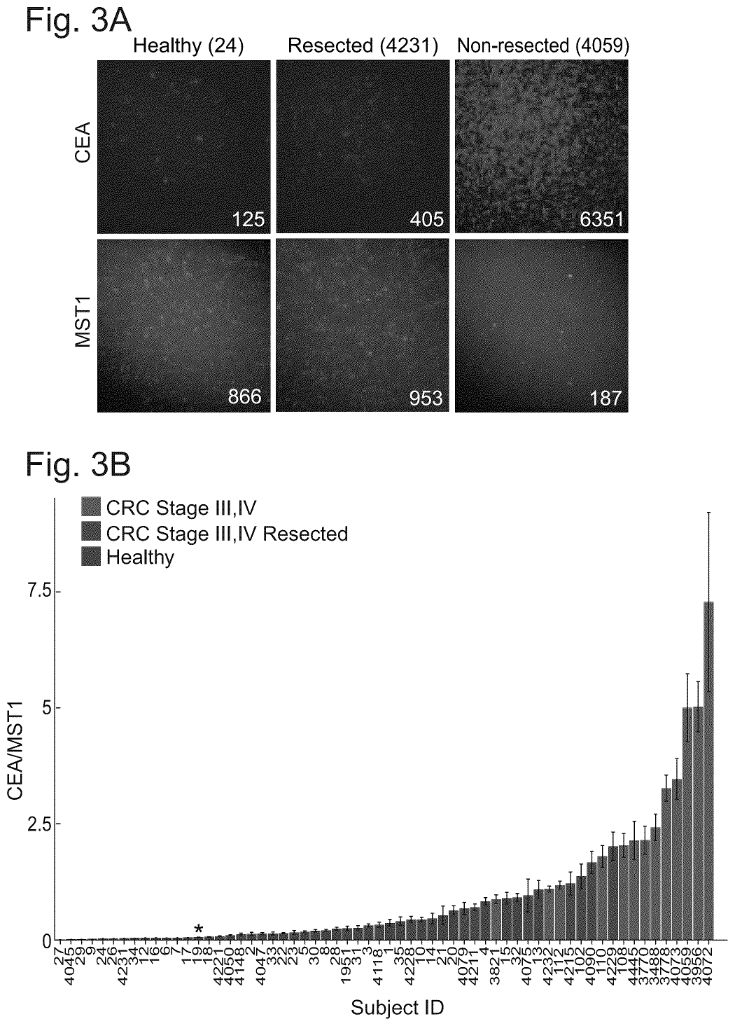

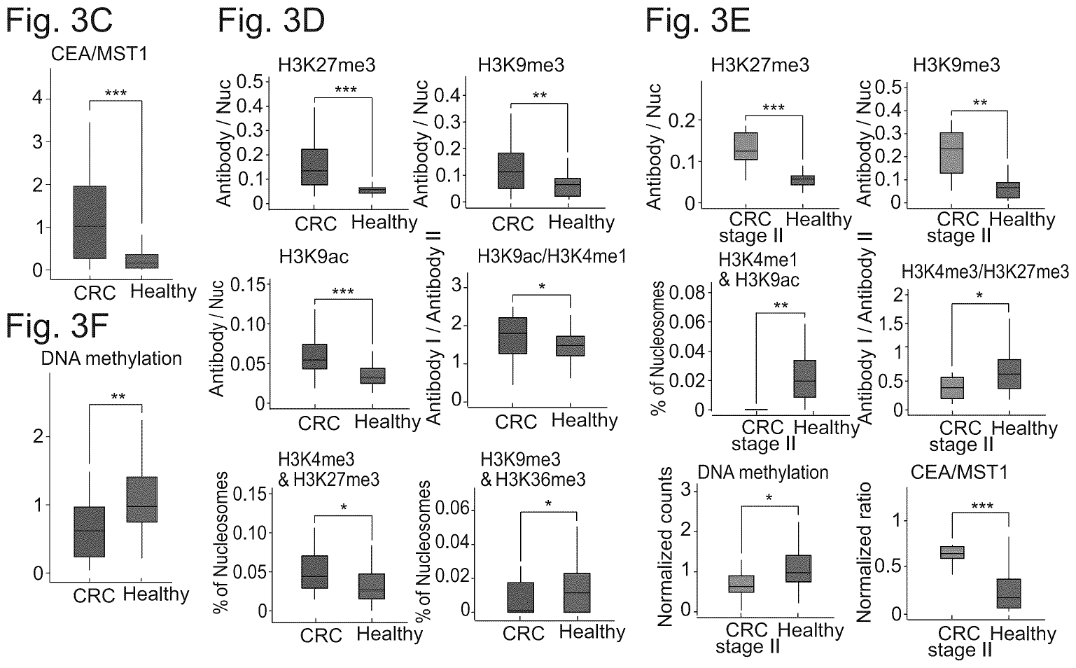

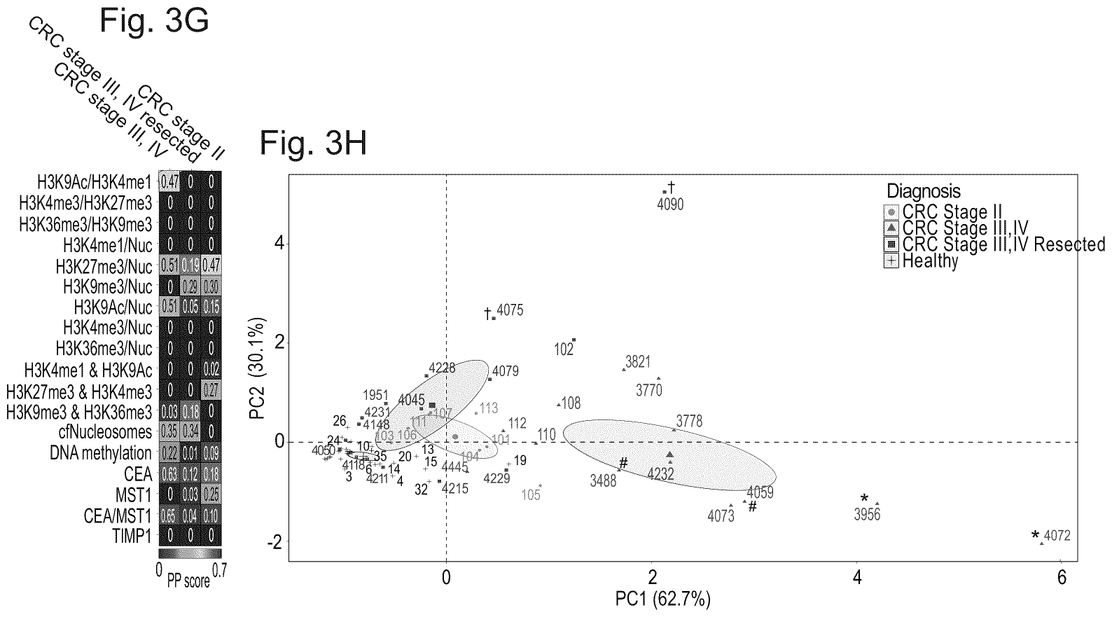

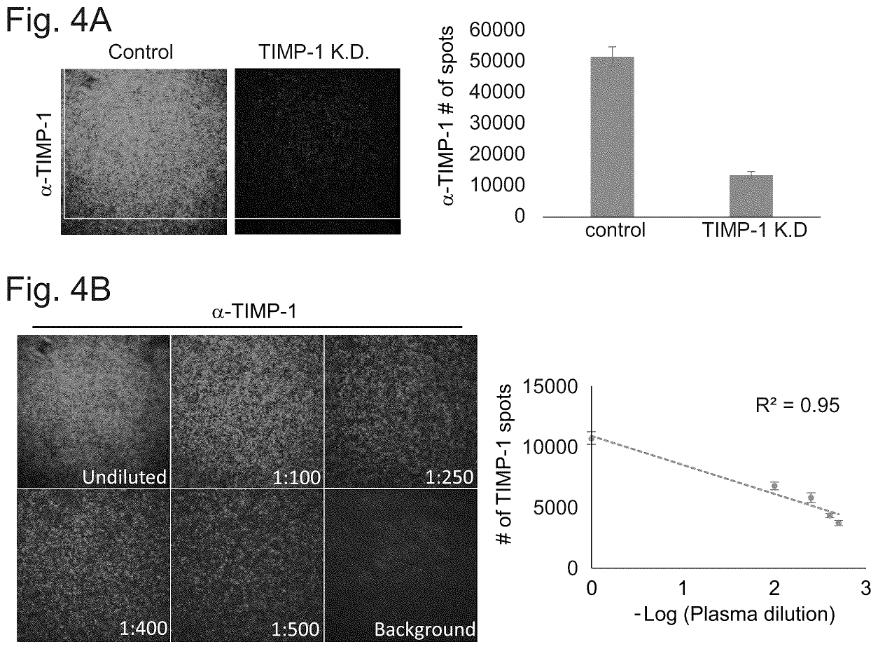

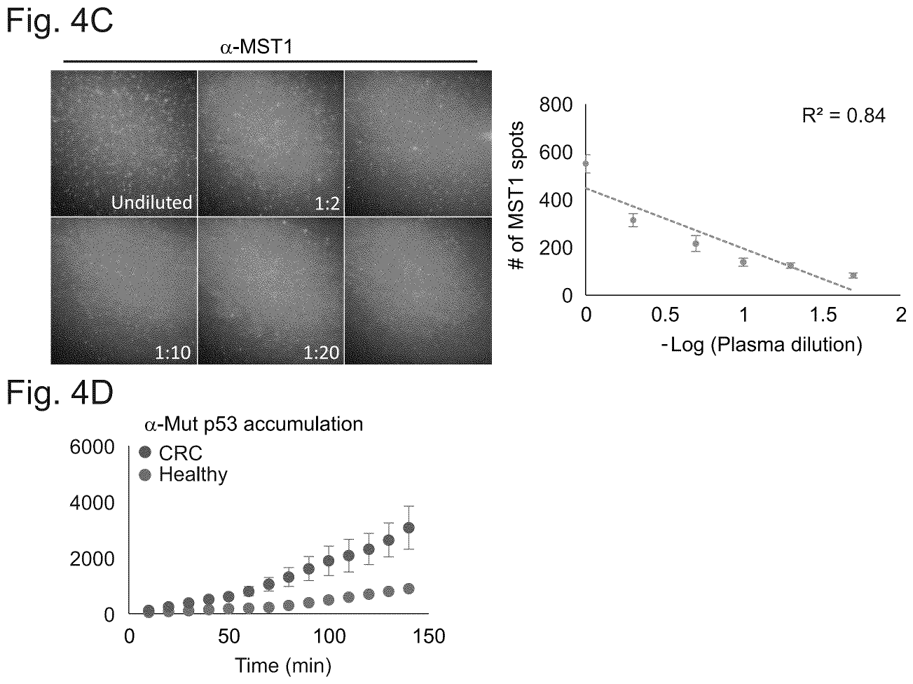

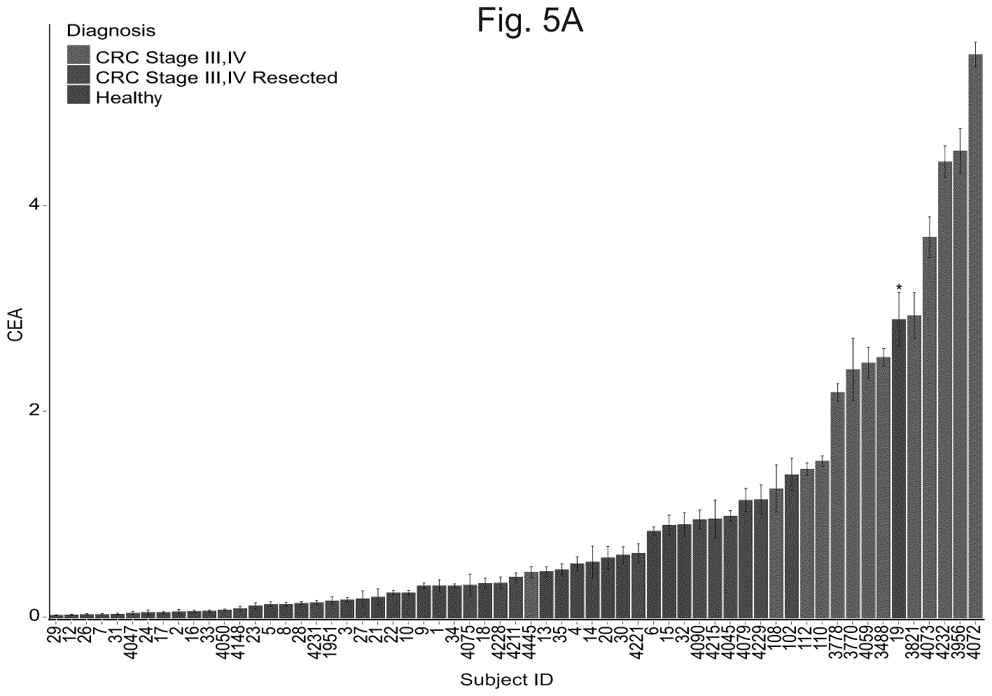

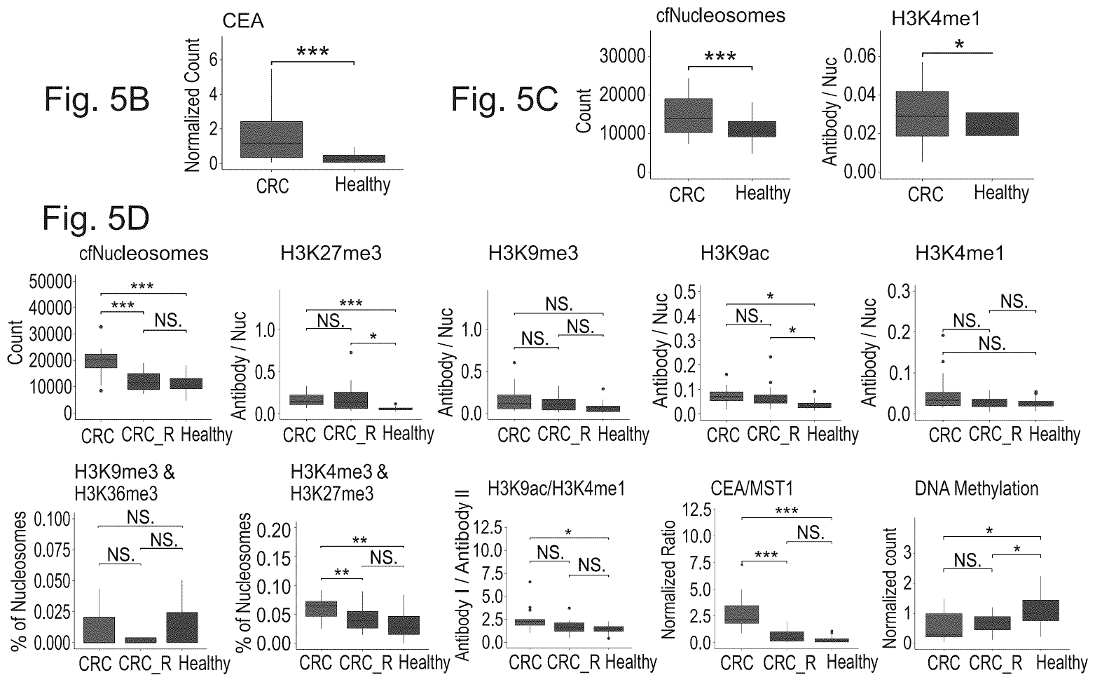

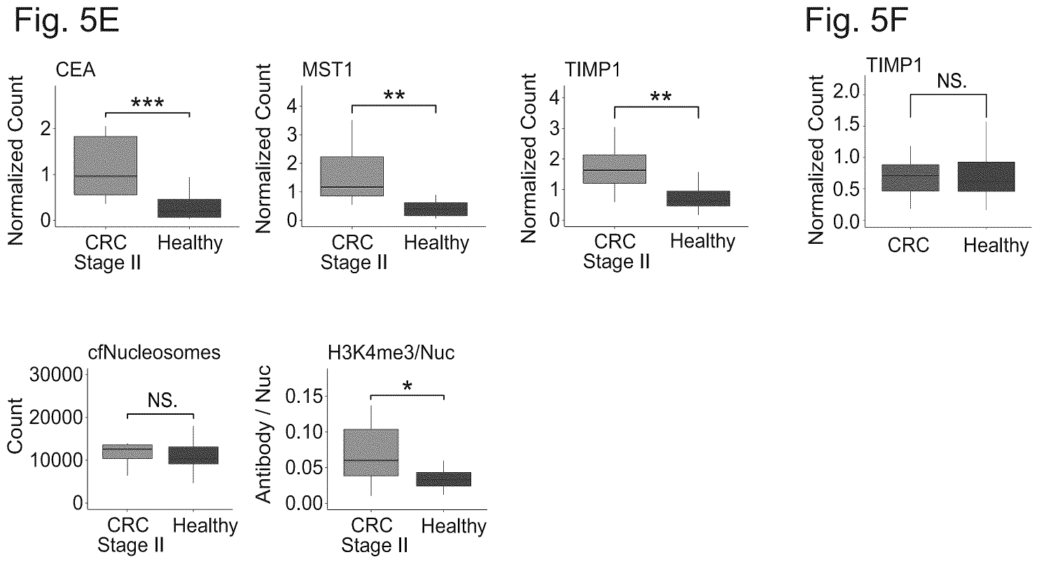

FIGS. 5A-F show analysis of histone PTMs, protein biomarkers and DNA methylation in the cohort of plasma samples from healthy and CRC subjects.
(a) CEA normalized levels in the plasma of CRC patients and healthy individuals. Each bar represents a subject, data is presented as the mean+/−s.d. of 50 FOVs per sample. Sample 19 (Healthy) denoted by *. (b) Box plot representation of the data in A (healthy=33, CRC=29). Box plots limits: 25-75% quantiles, middle: median, upper (lower) whisker to the largest (smallest) value no further than 1.5× interquartile range from the hinge. P values were calculated by Welch's t-test. *** P value <0.001. (c) Global level of cfNucleosomes, and levels of H3K4me1-modified nucleosomes, significantly differ between healthy and CRC late stage samples (healthy=33, CRC=29). P values were calculated by Welch's t-test. * P value <0.05 ** P value <0.01. *** P value <0.001. (d) Multiple comparison of all significant parameters between CRC, CRC resected (CRC_R) and healthy samples (CRC=13, CRC_R=16, healthy=33), corresponding to the data presented in main FIG. 3c,d,e. Of note, while all of these parameters differ between healthy versus the combined cohort of all CRC patients, this figure shows the differences between CRC patients with/without tumor resection. In some parameters, resected patients show higher similarity to healthy, and in other parameters, they are similar to CRC patients prior to tumor resection. See methods for P value calculation. * P value <0.05 ** P value <0.01. *** P value <0.001. (e) Levels of CEA, MST1 and TIMP1, as well as H3K4me3-modified nucleosomes, significantly differ between healthy and early stage CRC patients (healthy=33, early CRC=8). Total levels of cfNucleosomes do not show significant difference between the groups, likely due to low tumor burden at early stage patients. P values were calculated by Wilcoxon rank sum exact test. * P value <0.05 ** P value <0.01. *** P value <0.001. (f) TIMP1 levels do not significantly differ in the cohort of healthy versus late-stage CRC patients.

### DESCRIPTION OF SPECIFIC EMBODIMENTS OF THE INVENTION

The present invention, in some embodiments thereof, relates to methods and kits for analyzing nucleosomes and plasma proteins.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The analysis of cell-free DNA (cfDNA) in plasma represents a rapidly advancing field in medicine, providing information on pathological processes in the body. Blood cfDNA is in the form of nucleosomes, also referred to herein as “cfNucleosomes”, which maintain their tissue- and cancer-specific epigenetic state. However, their minute amount in body fluids demands the increase of assay sensitivity.

While developing a new assay modality for high sensitivity testing of cfNucleosomes by total internal reflection (TIRF) microscopy at the single molecule level, the present inventors devised a novel method for immobilization of cfNucleosomes on a solid support, which is followed by TIRF-based multiplex chromatin analysis and optionally sequencing. Using this method, the present inventors successfully managed to detect, in high-resolution (i.e., above 10,000 events per modification per sample, e.g., plasma <1 ml), six active and repressive histone modifications, their ratios and combinatorial patterns, on millions of individual nucleosomes by single-molecule imaging. In addition, it provides sensitive and quantitative data on plasma proteins, including detection of non-secreted tumor-specific proteins such as mutant p53.

Applying this analysis to a cohort of plasma samples detected colorectal cancer at high accuracy and sensitivity, even at early stages. Finally, combining it with direct single-molecule DNA sequencing revealed the tissue-of-origin of the tumor. A specific configuration of the method is termed “EPINUC” for Epigenetics of Plasma Isolated Nucleosomes that provides multi-layered clinical-relevant information from limited liquid biopsy material, establishing a novel approach for cancer diagnostics.

Thus, according to an aspect of the invention, there is provided a method of analyzing nucleosomes, the method comprising:
(a) isolating a plurality of nucleosome molecules from a biological sample;
(b) enzymatically linking adenine nucleotides to free DNA ends of the plurality of nucleosome molecules, wherein at least a portion of the adenine nucleotides comprises a label, such that the plurality of nucleosome molecules become attached to a labeled poly(A) tail;
(c) hybridizing the plurality of nucleosome molecules attached to the labeled poly(A) tail to a solid support coated with poly(T); and
(d) imaging the solid support, whereby the plurality of nucleosome molecules are visualized.

As used herein “chromatin” refers to a chromatic fragment, that is a segment of cell-free genomic DNA being in association with a nuclear protein. Exemplary chromatin fragments may be oligonucleosomes, mononucleosomes, centromeres, telomeres or genomic DNA bound by a transcription factor or chromatin remodeling factor.

The fundamental building block of chromatin is the nucleosome, composed of about 146 bp of DNA wrapped around an octamer of histone proteins, A nucleosome can include combinations of core histones and histone variants (Sarma, K. and Reinberg, D., 2005. Histone variants meet their match. Nature Reviews Molecular Cell Biology 6, 139-149). Histones are heavily modified by covalent attachment of various chemical groups at specific amino acid positions. These modifications are an integral component of the epigenetic control of genome function, enabling the manifestation of unique cellular phenotypes in multicellular organisms, which harbor identical genomic sequences.

According to a specific embodiment, the chromatin is circulating chromatin, also referred to as “cell-free chromatin” which are fragments that may comprise at least one nucleosome. The cell-free chromatin fragments may be mononucleosomes. The cell-free chromatin fragments may be a stretch of 2-5 adjacent nucleosomes.

According to a specific embodiment, the plurality of nucleosome molecules comprise cell-free nucleosomes (cfNucleosomes), which include oligonucleosomes or mononucleosomes.

As used herein “a biological sample” typically refers to a biological fluid.

According to a specific embodiment, the nucleosomes are human nucleosomes.

The biological sample may be from a subject or a patient in need thereof. The biological sample may be serum, plasma, lymph, blood, blood fractions, urine, synovial fluid, spinal fluid, saliva, circulating tumor cells or mucous. Not being bound by a theory, circulating chromatin and chromatin released from apoptotic cells are already fragmented to oligonucleosomes and mononucleosomes, thus not requiring digestion with a nuclease.

According to a specific embodiment, the biological sample is a plasma sample.

The sensitivity of the assay described herein allows analysis of cell-free chromatin in limited amounts of the sample, such as below 1 ml plasma.

Biological fluids can be collected using methods which are well known in the art.

For example, for plasma collection, whole blood is transferred into commercially available anticoagulant-treated tubes e.g., EDTA-treated or citrate-treated. Heparinized tubes may also be used. Cells are removed from plasma such as by centrifugation (e.g., 10 minutes at 1,000-2,000×g) using a refrigerated centrifuge. According to an embodiment, centrifugation for 15 minutes at 2,000×g depletes platelets in the plasma sample. The resulting supernatant is designated plasma. Following centrifugation, the liquid component (plasma) is transferred into a clean polypropylene tube. The samples should be maintained at 2-8° C. while handling. Alternatively, plasma can be aliquoted and stored at −20° C. or lower e.g., −80° C.

It will be appreciated that plasma DNA (cfNucleosomes) can be used without further isolation. However, cell free (cf) DNA can be extracted using methods which are well known in the art or while employing dedicated kits e.g., Mag-Bind cfDNA Kit (Omega Bio-Tek, M3298-01). For optimized yield, the protocol can be modified by increasing elution time to 20 minutes on a thermomixer, at 1,600 rpm, in 15 μl elution buffer at room temperature. Sample concentration is measured using a Fluorometer (e.g., Thermo Fisher Scientific).

As used herein “isolating”, “isolated”, “purified” refers to depletion of non-chromatin materials, such as cells, cell debris, plasma proteins and the like such that the chromatin or nucleosome is in a form that is substantially free (at least 60% free, preferably 75% free, and most preferably 90% free) from other components normally present with the chromatin or nucleosome in a native environment (e.g., cells, lipids, etc.).

Once the nucleosomes are at hand, their free DNA end is subjected to an enzymatic reaction, which comprises enzymatically linking adenine nucleotides to free DNA ends of the molecules. As least a portion of the adenine nucleotides comprises a label. In this manner the nucleosomes are modified to comprise a labeled poly(A) tail.

It will be appreciated, that the method as described herein is simple in that there is no need to include also a capture molecule such as biotin for binding to the surface, the polyA tail will perform this function. In addition, it is worth noting that according to the present teachings, the polyA tail is synthesized on the nucleosome de novo, i.e., there is no addition of a pre-made oligonucleotide polyA tail. Without being bound by theory, this distinction is important since it allows imaging the surface without washing labeled oligonucleotides (not linked to the nucleosomes) that may compete with the labeled nucleosomes on binding to the surface.

Typically, enzymatic linking comprises the use of a template-dependent DNA polymerase and a Terminal deoxynucleotidyl Transferase (TdT).

As used herein “template (DNA)-dependent DNA polymerase” refers to any DNA polymerase such as Klenow polymerase, polymerase I (Pol I), T4 DNA polymerase, Pfu polymerase and the like. According to a specific embodiment, the DNA polymerase is Klenow polymerase, also referred to as “Klenow fragment (3′→5′ exo-)”.

The exonuclease activity of Pol I limits its use in molecular experiments because the 5′-3′ exonuclease activity might interfere with generating single-stranded DNA for downstream applications. These different functions were successfully divided by separating Pol I into two fragments. The small fragment contains the 5′-3′-exonuclease activity while the large fragment or Klenow fragment retains the 5′-3′ polymerase and 3′-5′ proofreading activities. Thus, the Klenow fragment is very useful in double-stranded DNA synthesis (second strand cDNA synthesis), blunting by filling of 3′ ends (after restriction digestion or just filling in of large gaps), primer labeling (radiolabeled nucleotides onto 3′ ends), and DNA sequencing experiments.

Template-dependent DNA polymerases are commercially available such as from New England Biolabs (NEB). The concentration and conditions for using the enzymes are known to the skilled artisan and typically included in manufacturers' instructions.

Typically, added to the reaction mixture is a Terminal deoxynucleotidyl transferase (TdT). Terminal deoxynucleotidyl transferase (TdT) is also known as DNA nucleotidylexotransferase (DNTT) or terminal transferase. TdT catalyses the addition of nucleotides to the 3′ terminus of a DNA molecule. Unlike most DNA polymerases, it does not require a template. The preferred substrate of this enzyme is a 3′-overhang, but it can also add nucleotides to blunt or recessed 3′ ends. Further, TdT is the only polymerase that is known to catalyze the synthesis of 2-15 nt DNA polymers from free nucleotides in solution in vivo.

It will be appreciated that the TdT can also function on a 3′ overhang thereby negating the use of the DNA-dependent DNA polymerase.

TdT is commercially available such as from Takara Bio, Sigma Aldrich, Thermo Fisher, Enzymatics and more.

As mentioned, the synthesis of the poly adenosine tail is typically performed in the presence of labeled adenosine nucleotides (dATP). Hence, the reaction mixture comprises a combination of labeled and non-labeled nucleotides (e.g., 1:100 to 1:1000).

According to a specific embodiment, the dATP is fluorescently labeled, such as with a fluorophore. The label is used to detect a single chromatin (e.g., nucleosome) on the surface.

Fluorescent dyes for use in fluorescent microscopy are known in the art. Exemplary dyes may be any available fluorescent dye (see for instance WO2017034970). In preferred embodiments, dyes are chosen with distinguishable emission spectra and that have excitation spectra compatible with the laser present on the microscope.

According to a specific embodiment, the dye is selected from the group consisting of Alexa 488, Alexa 555, Alexa 640, CY3, CY5, an Atto Dyes, and a Pacific Dye.

According to a specific embodiment, the dye is Cy3.

The reaction mixture typically includes additional components such as buffer (typically of the enzyme, e.g., TdT), CoCl₂, MnCl₂, and T4 Polynucleotide Kinase.

An exemplary embodiment of the reaction mixture is provided in the examples section hereinbelow.

For nucleosome sequencing, as further described hereinbelow, ddATP is added (e.g., SIGMA, GE27-2051-01).

Following incubation, samples are inactivated such as by addition of EDTA (e.g., Invitrogen, 15575-038). Next, DNA is purified by AMPure SPRI beads (Beckman Coulter, A63881), and quantified by Qubit (Thermo Fisher Scientific).

To analyse the labeled nucleosomes, the labeled nucleosomes are contacted with a poly thymidine (polyT)-coated solid support. Not being bound by a theory, the solid support needs to be stable enough so that the DNA from the isolated chromatin fragments remains bound throughout the sequencing process. Any method of functionalizing a surface as known in the art is possible.

According to a specific embodiment, the solid support is coated with polyethylene glycol (PEG). Not being bound by a theory, a PEG-coated surface prevents non-specific binding of labels to the solid support. According to a specific embodiment, the surface or the reaction mixture does not comprise biotin or avidin or any derivation thereof.

Not being bound by a theory, the surface binds labeling ligands non-specifically and needs to be blocked. In a preferred embodiment the solid support is blocked with spermine or BSA.

The solid support may comprise slides, arrays, channels, beads, bubbles, and the like that contain polyT and possibly PEG. In one embodiment, a flow cell houses the solid support. The solid support may comprise glass or fused silica slide. Other solid supports contemplated herein include, but are not limited to, polydimethylsiloxane (PDMS), silicon, polystyrene, polycarbonate, polyvinylchloride, polymethyl methacrylate, cyclic olefin copolymer, or a combination thereof. Suitable dimensions of a flow channel include a width in the range of 0.025 mm to 10 mm, 1 mm to 6 mm, and 2 mm to 4 mm; a length in the range of 0.1 mm to 10 mm, 0.5 mm to 5 mm, and 1 mm to 3 mm; and a height in the range of 0.001 mm to 2 mm, 0.001 mm to 1 mm, and 0.01 mm to 1 mm. According to a specific embodiment, the width range is 0.05 mm to 0.5 mm. The present invention can be used with any surface compatible with fluorescence microscopy, preferably TIRF, that is functionalized with polyT and possibly PEG. Methods of binding a polyT to a glass surface are known in the art. All three types of functionalized glass slides (amine, aldehyde and epoxy) may be used.

Contacting is performed to allow cannonical base complementation resulting in hybridization between adenosine (A) and thymidine (T).

According to a specific embodiment, the Hybridization is performed at room temp, for about 10 min using an imaging buffer [e.g., IMB: 12 mM, 440 HEPES pH 8 (Thermo Fisher Scientific, 15630056), 40 mM TRIS pH 7.5 (Gibco, 115567-027), 441 60 mM KCL (SIGMA, 60142), 0.32 mM EDTA (Invitrogen, 15575-038), 3 mM MgCl2 (SIGMA, 442 63069), 10% glycerol (Bio-Lab, 56815), 0.1 mg/ml BSA (SIGMA, A7906) and 0.02% Igepal 443 (SIGMA, I8896).

The support may be washed or not (to remove unbound nucleotides and other components of the reaction), and then imaged such that the plurality of the nucleosomes are visualized at the single molecule level.

Fluorescent microscopy may be used to visualize the cfNucleosomes. Not being bound by a theory, fluorescent microscopy would produce background from unbound labeling ligands present in solution, but the fluorescence signal of bound isolated chromatin fragments may be distinguishable. In a preferred embodiment, TIRF microscopy is used. TIRF microscopy enables a selective visualization of surface regions, thus background may be eliminated, TIRF microscopy may utilize one laser, two lasers, three lasers or four lasers. Simultaneous multicolor detection of 2-4 dyes may be performed (as for the combinatorial imaging, see below). The dyes may be excited by a single laser and emit a different wavelength. The dyes may have different fluorescent lifetimes. The dyes may be excited by different lasers and emit different wavelengths. Dyes and lasers applicable to TIRF microscopy are known in the art. The microscope may be set up in any configuration as described by Harris et al. in PCT publication WO2006055521, TIRF single molecule analysis and method of sequencing nucleic acids. Additional methods for imaging single molecules are described by Friedman L J, Chung J, and Gelles J. (Viewing dynamic assembly of molecular complexes by multi-wavelength single-molecule fluorescence. Biophys J. 2006 Aug. 1; 91(3): 1023-31. Epub 2006 May 12) and in PCT publication number WO2006133221, Apparatus and method for introducing multiwavelength laser excitation in fluorescence microscopy, incorporated herein by reference.

TIRF microscopy enables detection of molecules or events that occur close to a solid surface, where an evanescent wave excites fluorophores. It provides a powerful means for detecting single fluorescent molecules that are within −100 nm of a surface and separated from each other by the diffraction limit (˜200 nm). In preferred embodiments, the present invention leverages TIRF microscopy to decode combinatorial modification states of hundreds of millions of nucleosomes captured on solid surface (see WO2017034970).

Once, information on the presence and/or level of nucleosomes in the sample and/or position on the slide is obtained and preferably stored (in any computed associated memory device), the sample may be subjected to a treatment which cleaves the fluorophore.

Thus, according to a specific embodiment, the method further comprises cleaving the label and optionally washing it prior to further analysis e.g., step (e) of analyzing a target molecule, as detailed below.

Nucleosome positions are recorded using a TIRF microscope, which excites and detects fiuorophores in a thin region and allows single-molecule quantification on planar surfaces. The TIRF microscope used includes two lasers, 532 nm/75 mW and 640 nm/40 mW. After positions are determined, the fluorophore is cleaved and washed away.

More specifically, experiments are performed using a TIRF microscope with two lasers, 532 nm/75 mW and 640 nm/40 mW, for fluorescence excitation (Compass 215M, Cube-40C, Coherent). Both laser beams are filtered through band pass filters (Chroma) and spectrally separated by a dichroic mirror (T:640 nm, R:532 nm). They then pass through the TIRF lens and total internal reflection is achieved through a 60×TIRF oil objective with index of refraction 1.49 (Nikon), and imaged onto a CCD camera. After imaging of nucleosomes, the fluorophore is cleaved via addition of TCEP (Bond-Breaker™ TCEP Solution, ThermoFisher Scientific, 77720) diluted 1:10 in imaging buffer. All positions are imaged again in order to discard residual spots from further analysis.

It will be appreciated that the polyA fluorophore (e.g., Cy3) need not be cleaved but rather further imaged together with the target molecules as further described hereinbelow (e.g., dependent on the number of label to the target molecules to be detected at the later stage), provided different fluorophores (makers) are used for labeling the target molecules. Simultaneous detection of the target molecules and the nucleosomes provides accurate multiplex data.

Regardless, the above described already provides insight regarding the total number of nucleosomes in the sample. It will be appreciated that the total number of nucleosomes in the plasma correlates with cancer, whereby an increase in the number as compared to a normal sample of a healthy (non-cancerous) subject has a diagnostic value, i.e., is indicative of cancer, as further detailed below.

In order to gain further insight regarding the nucleosome composition in the sample inferred from an analysis at the single molecule level, the method further comprises in embodiments thereof:
(e) incubating the solid support with at least one labeling ligand with specific binding affinity for a target molecule of said nucleosome and wherein the labeling ligand includes a marker;
(f) imaging the solid support, whereby said plurality of nucleosome molecules comprising said target molecule are visualized.

As used herein “a target molecule” refers to a molecule which is attached to the DNA portion of the nucleosome (or chromatin).

According to a specific embodiment, the target molecule is a nucleotide modification.

According to a specific embodiment, the nucleotide modification is selected from the group consisting of 5-methyl-(5-mC), 5-hydroxymethyl-(5-hmC), 5-formyl-(5-fC) and 5-carboxy-(5-eaC) cytosine.

According to a specific embodiment, the nucleotide modification is 5-methyl-(5-mC), 5-hydroxymethyl-(5-hmC).

According to a specific embodiment, the target molecule is a post translational modification.

According to a specific embodiment, the target molecule is a histone modification and/or a histone variant.

The term “modified histone” or “histone modification” refers to a histone protein, wherein one or more of the amino acid residues have been modified post-translationally. Examples of post translation modifications include, but are not limited to, histone modifications including lysine mono-, di- and tri-methylation, lysine acetylation, Arginine mono-methylation and symmetric or asymmetic di-methylation, citrullination, ubiquitinylation, serine or threonine phosphorylation and proline isomerization. It will be appreciated by those skilled in the art that these and other histone posttranslational modifications can exert activating or repressive effects on gene expression. Histone proteins include, but are not limited to H1, H2A, H2B, H3, H4, and any variants thereof. Histones are typically modified at the last 30 amino acid residues of the amino terminus.

According to a specific embodiment, the histone variant is selected from the group consisting of macroH2A1.1, macroH2A1.2, H2AZ, H2AX, H3.1 and H3.3.

According to a specific embodiment, the histone modification is selected from the group consisting of acetylation, methylation, phosphorylation, ribosylation, citrullination, ubiquitination, hydroxylation, glycosylation, nitrosylation, glutamination and isomerisation.

Thus, the target molecule may comprise a histone modification, nucleotide modification, histone variant, chromatin remodeling factor, a methyl-transferase, an acetylase, a deacetylase, a kinase, a phosphatase, a ubiquitin ligase or a transcription factor.

According to a specific embodiment, the histone modification is selected from the group consisting of H2B Ser 14 (Phos), H3 Ser 10 (Phos), H3 Lys 9 (Me), H3 Lys 27 (Me), H3 Lys 36 (Me), H3 Lys 79 (Me), H4 Lys 20 (Me), H3 Lys 4 (Me), H3 Lys 9 (Ac), H3 Lys 14 (Ac), H3 Lys 23 (Ac), H4 arg 3 (Me), H3 Lys 27 (Ac), H4 arg 3 (Me), H4 lys 5 (Ac), H4 Ser 2 (phos), H4 Arg 3(me), H4 Lys 5 (Ac) and H3 Lys 18 (Ac).

According to a specific embodiment, the histone is histone H3.

According to a specific embodiment, the modification is selected from the group consisting of H3K9me3, H3K27me3, H3K4me3, H3K36me3, H3K9ac and H3K4me1.

Each of these modifications can be analyzed per se at the single molecule level, or a combination of modifications on a single molecule is analyzed.

According to a specific embodiment, the marker is a fluorophore.

According to a specific embodiment, the labeling ligand may be an antibody or an antibody fragment.

The term “antibody” is used interchangeably with the term “immunoglobulin” herein, and includes intact antibodies, fragments of antibodies, e.g., Fab, F(ab′)2 fragments, and intact antibodies and fragments. The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein “specific binding” of an antibody means that the antibody exhibits appreciable affinity (e.g., at the uM to nM range) for a particular antigen or epitope and, generally, does not exhibit significant crossreactivity.

Any of the markers may be a fluorescent marker. In preferred embodiments, markers imaged at the same time may emit different wavelengths. The fluorescent dyes may have different fluorescent lifetimes. In preferred embodiments, the dyes are selected from Alexa 488, Alexa 488, Alexa 555, Alexa 640, CY3, CY5, an Atto Dye, or a Pacific Dye. In a more preferred embodiment, the labeling ligand comprises Alexa 488 and Alexa 647 and optionally the nucleosomes are marked with Cy3.

Detection antibodies are labeled using, for example, Alexa flour antibody labeling kits (Thermo Fisher Scientific, A20181/A10237/A20186) according to the manufacture protocol. Labeled antibodies may be purified such as by size exclusion chromatography such as by using Bio-Spin 6 columns (Bio-Rad, 7326200) followed by measurement of protein concertation using Nanodrop 2000 at 260 nm.

Antibodies to the target molecules are well known in the art and are commercially available such as by Abcam.

Exemplary antibodies which can be used according to some embodiments of the invention are provided hereinbelow in the Examples section which follows.

For example, one may analyze the presence of combinations of markers simultaneously in a number of aliquots of a specific biological sample, whereby each aliquot (or channel) is contacted with labeling ligands, e.g., antibodies (e.g., 2-3) for a specific combination of target molecules. Alternatively, a sequential detecting may be performed, whereby a first pair of modifications is analyzed, followed by removing and washing the labeling ligands, incubation with a second pair of a labeling ligand followed by removing and washing the labeling ligands and incubation with a third pair. This may be repeated with any pair, triplet, quadruplet and more.

According to a specific embodiment, the antibodies target the tri-methylations on histone H3 lysine 9 (H3K9me3) and lysine 27 (H3K27me3), associated with gene silencing and heterochromatin, as well as antibodies targeting marks associated with active transcription: tri-methylation of histone H3 on lysine 4 (H3K4me3) and lysine 36 (H3K36me3), and acetylation on lysine 9 (H3K9ac). In addition, the panel may include an antibody targeting mono-methylation of histone H3 on lysine 4 (H3K4me1), a mark associated with enhancers.

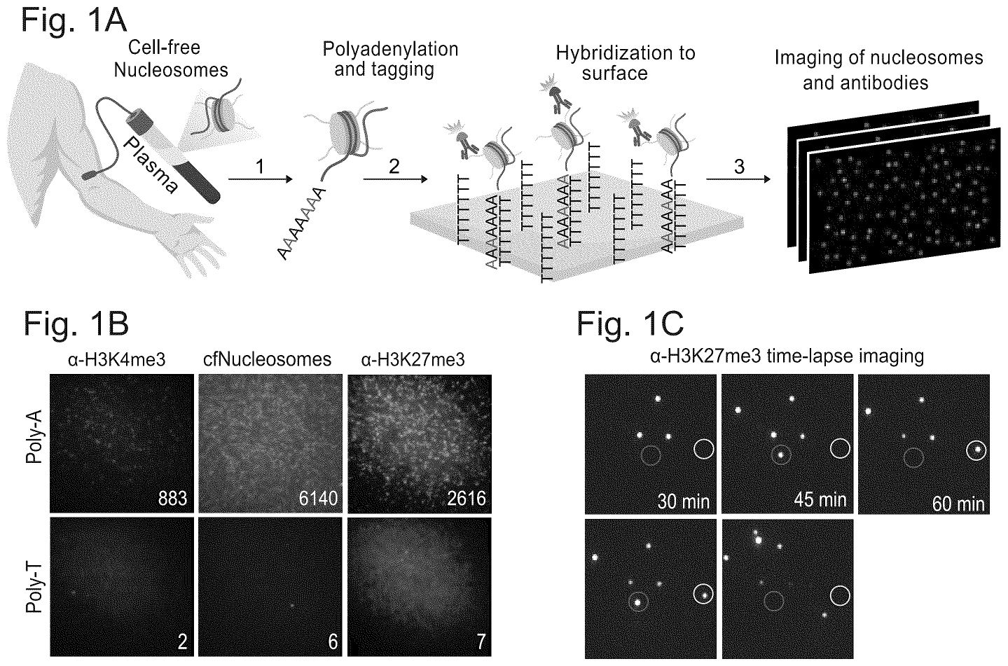

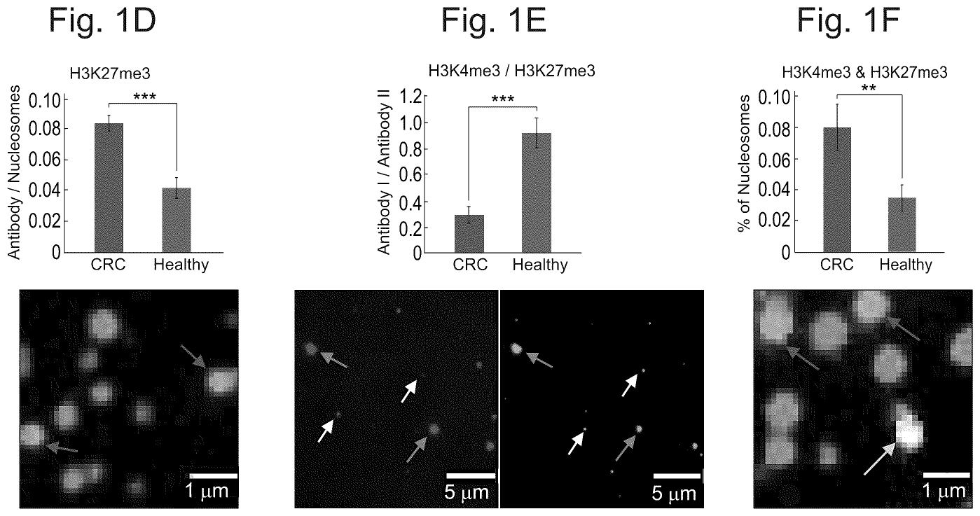

Thus, for example, three pairwise combinations of antibodies may be used. Thus, multi-parametric data can be obtained for six histone PTMs, comprising of the percentage of modified nucleosomes in each sample, the ratio between various histone modifications, and the percentage of nucleosomes that harbor a combinatorial pattern of two modifications (e.g., FIGS. 1d,e,f). It will be appreciated that the present teachings present the only technology that enables counting of multiple histone PTMs, as well as combinatorially-modified nucleosomes, at a single-molecule precision, from low volume plasma sample (<1 ml).

Exemplary combination sets include, but are not limited to:

$$H 3 K 4 me 1 + H 3 K 9 ac \\ H 3 K 27 me 3 + H 3 K 4 me 3 \\ H 3 K 9 me 3 + H 3 K 36 me 3$$

The imaging of any of the target molecules and/or labeled polyA tails may comprise imaging the markers/label at more than one time point. The imaging may comprise imaging the markers/label in a time-lapse fashion. For example, up to 150 images may be taken over a period of up to 1.5 hours. Real time imaging (measuring association-dissociation events of the antibodies) negates the need to wash the label/marker prior to or during the imaging. However, many more images can be taken.

According to a specific embodiment, the marker is a label e.g., fluorescent label which comprises a fluorophore. According to a specific embodiment, the label is distinctive of the label used in the polyA tail. According to a specific embodiment, the label comprises a plurality of different labels each for a distinct target molecule, when multiplex detection is done.

In an exemplary embodiment, the labeling ligands (e.g., antibodies) are typically diluted in imaging buffer such as described above and images are taken every 10-20 minutes for a total incubation time of 1.5, 2, 3, 4, 5, 6, 7, 8, 9, 10, 11, 12, 13, 14, 15, 16, 17, 18, 19, 20, 22 or 24 hours.

Time-lapse imaging of different labeling ligands (e.g., antibodies) can be performed sequentially or simultaneously (for example, green-labeled and red-labeled antibodies can be imaged simultaneously with the two lasers, or one antibody can be imaged for e.g., up to 24 hours or until a plateau is detected.

According to a specific embodiment, 3 channels are simultaneously imaged for each field of view, for example, antibodies to target molecules at the red and cyan channel and cfNucleosomes at the green channel, as an exemplary configuration.

Image analysis may be performed with CellProfiler cell image analysis software, free open-source software (www(dot)cellprofiler(dot)org/). The pipeline for image analysis is available upon request. Briefly, image analysis is done in three steps: (1) Time-lapse images of antibody binding events are aligned and stacked, and all binding events are summed (spots are also assigned a number that corresponds to the number of binding events per nucleosome). (2) Stacked images are. Only binding events that align with nucleosomes are filtered and saved for further analysis. Co-localization events (of 2 antibodies) are defined as combined modifications.

In each experiment millions of nucleosomes can be visualized. The TIRF microscope captures only target-bound antibodies while eliminating background from un-bound antibodies in solution. The ability to monitor antibody binding and dissociation events temporally allows for quantifying two histone marks simultaneously regardless of any potential steric hindrance. After detection of the first two antibodies, Applicants wash the flow cell and re-image to exclude remaining binding events (˜2%). Next, a second pair of antibodies is added and imaged (FIGS. 1A-F).

It will be appreciated that the imaging of step (f), that is imaging the labeling ligand to the target molecule is done by TIRF microscopy but also the step of imaging the label on the polyA tail can be done (but not necessarily) with TIRF microscopy.

Once the data is obtained and stored, the solid support may be treated as needed for sequencing the DNA of the imaged nucleosomes.

Thus, according to a specific embodiment, the methods described herein may further comprise sequencing the DNA portion of the plurality of nucleosome molecules.

The solid support is washed to remove the labeling ligands and DNA sequencing is performed as known in the art such as by using Helicos True Single Molecule Sequencing (www(dot)seqll(dot)com/)²¹⁴⁵. FluoSpheres preparation: FluoSpheres (Carboxylate-Modified Microspheres, Thermo Fisher Scientific, F8789) are conjugated to dA50-amine (IDT), tailed as previously described, and hybridized to the surface to serve as reference points for stage drift correction during alignment of sequencing images.

Appropriate modifications are taken to perform single-Molecule Hydroxymethylation and 5-mc sequencing on the surface.

The present teachings can be harnessed for research and diagnostic applications.

As used herein the term “diagnosing” refers to determining presence or absence of a pathology (e.g., a disease, disorder, condition or syndrome), classifying a pathology or a symptom, determining a severity (i.e., staging) of the pathology, monitoring pathology progression, forecasting an outcome of a pathology and/or prospects of recovery and screening of a subject for a specific disease.

Thus, according to an aspect of the invention there is provided a method of diagnosing a disease associated with modified, cell-free nucleosomes (cfNucleosomes) comprising: analyzing nucleosome molecules in a biological fluid as described herein, wherein presence of a pathological nucleorise phenotype is indicative of a disease associated with modified cfNucleosomes.

As used herein “disease associated with modified, cell free nucleotosomes” refers to a disease in which a statistically significant higher cfNucleosomes concentration and/or higher levels of modified cfNucleosomes is indicated.

Nucleosomes can be detected in the serum of healthy individuals (Stroun et al., Annals of the New York Academy of Sciences 906: 161-168 (2000)) as well as individuals afflicted with a disease state. Moreover, the serum concentration of nucleosomes is considerably higher in patients suffering from benign and malignant diseases, such as cancer and autoimmune disease (Holdenrieder et al (2001) Int J Cancer 95, 114-120, Trejo-Becerril et al (2003) Int J Cancer 104, 663-668; Kuroi et al 1999 Breast Cancer 6, 361-364; Kuroi et al (2001) Int j Oncology 19, 143-148; Amoura et al (1997) Arth Rheum 40, 2217-2225; Williams et al (2001) J Rheumatol 28, 81-94). Not being bound by a theory, the high concentration of nucleosomes in tumor bearing patients derives from apoptosis, which occurs spontaneously in proliferating tumors. The presence of elevated levels of nucleosomes in the blood of patients can serve as a diagnostic of diseases associated with enhanced cell death (Holdenrieder et al., Anticancer Res, 19 (4A): 2721-2724 (1999)). Nucleosomes circulating in the blood contain uniquely modified histones, wherein the unique histone epitope and/or the associated DNA can be correlated with a particular disease state. For example, U.S. Patent Publication No. 2005/0069931 relates to the use of antibodies directed against specific histone N-terminus modifications as diagnostic indicators of disease, employing such histone-specific antibodies to isolate nucleosomes from a blood or serum sample of a patient to facilitate purification and analysis of the accompanying DNA for diagnostic/screening purposes. Accordingly, the present invention may be used for the combinatorial single molecule analysis of cell-free mono or oligonucleosomes. The identification of modified histones and the associated DNA of single chromatin fragments can serve as diagnostic, prognostic or disease monitoring markers of disease and congenital defects. In another embodiment, the presence and/or percentage of bivalent nucleosomes can serve as diagnostic markers of disease and congenital defects.

Examples of such medical conditions which are associated with modified, cell-free nucleosomes, include, but are not limited to, pre-malignant and malignant neoplasms, histocytoma, glioma, astrocyoma, osteoma, lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma, leukemias, systemic lupus erythematosus, psoriasis, bone diseases, fibroproliferative disorders of connective tissue, cataracts and atherosclerosis. The diagnostic method may be used to detect cancer or risk for cancer. The biological sample may be from a subject identified to be suffering from or at risk for developing cancer. The cancer may be histocytoma, glioma, astrocyoma, osteoma, lung cancer, small cell lung cancer, gastrointestinal cancer, bowel cancer, colon cancer, breast carcinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma or leukemia.

According to a specific embodiment, the disease is sepsis.

According to a specific embodiment, the disease is lupus.

According to a specific embodiment, the disease is Parkinson's disease.

According to a specific embodiment, the disease is cancer or a pre-malignant lesion.

According to a specific embodiment, the cancer is lung cancer or colorectal cancer.

According to a specific embodiment, the cancer is colorectal cancer.

According to a specific embodiment, the phenotype is selected from the group consisting of:
(i) increased concentration of nucleosomes as compared to same in a non-cancerous biological fluid;
(ii) altered percentage of modified nucleosome in a specific target molecule in the biological fluid as compared to same in a non-cancerous biological fluid;
(iii) altered ratio between a plurality of said target molecules as compared to same in a non-cancerous biological fluid;
(iv) altered percentage of nucleosomes that comprise a combinatorial pattern of target molecules in a single nucleosome as compared to same in a biological fluid of a non-cancerous biological fluid.

As used herein “increased” refers to an increase of at least 10%, 20%, 30%, 40%, 50% and more (e.g., about 34%) in a statistical significant manner as compared to same examined parameter in the indicated control.

As used herein “altered” or “alteration” refers to a shift (increase or decrease) of at least 2 fold, 3 fold, 4 fold, 5 fold, 7 fold, 10 fold, 15 fold or at least 50 fold in a statistical significant manner as compared to same examined parameter in the indicated control.

The comparison is empirically done or relies on pre-obtained data (i.e., control).

The control is of the same fluid, under the same conditions from a known sample which is not inflicted with the disease.

Alternatively or additionally the control can be of the same subject being tested at another disease stage (e.g., absent of the disease or prior to or following treatment).

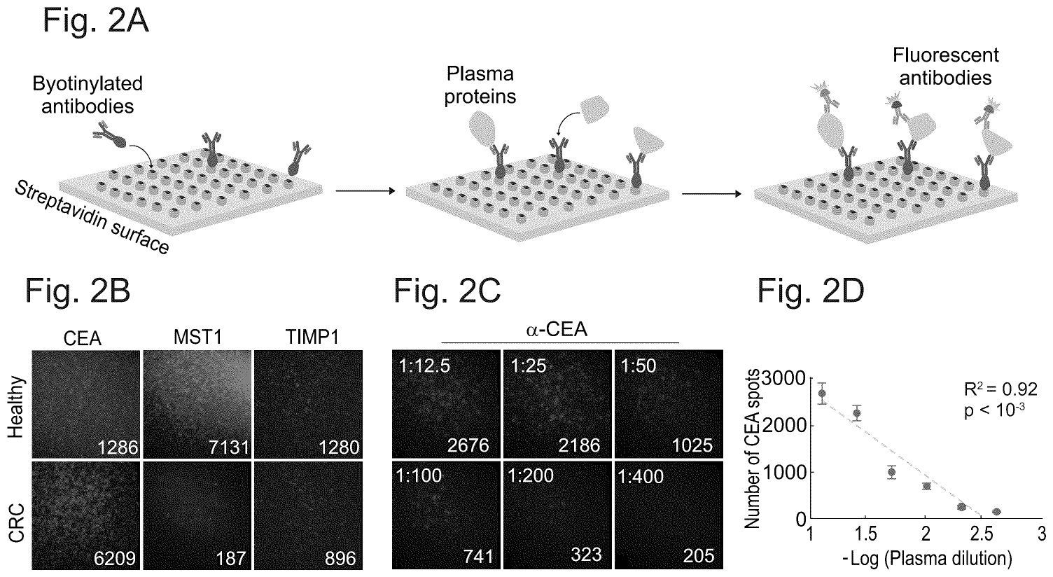

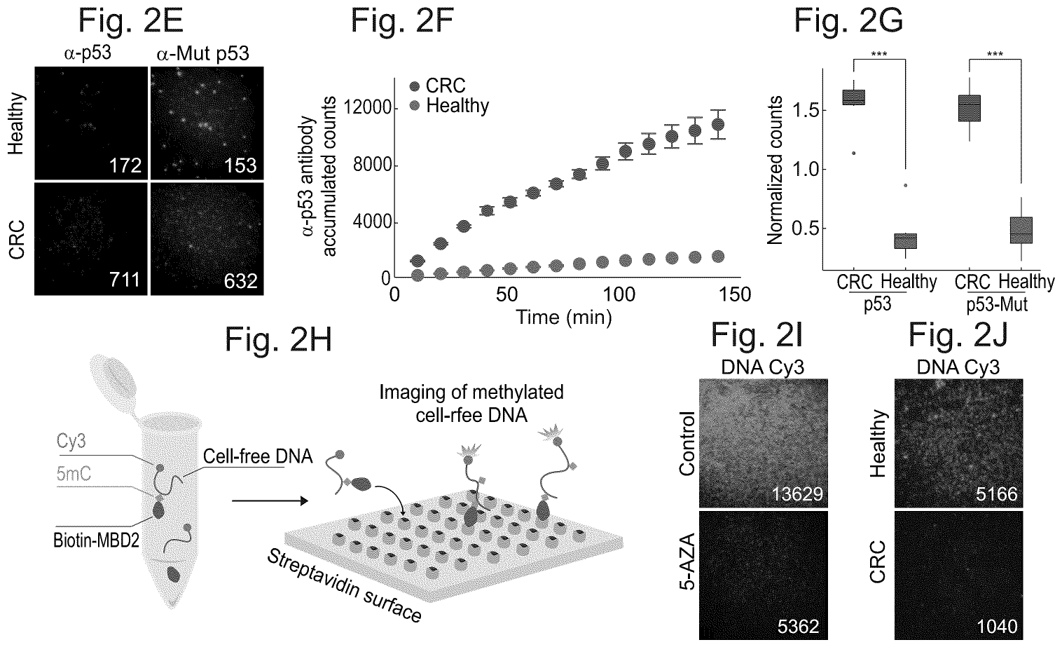

As shown in the examples section which follows, colorectal cancer (CRC) patients had higher levels of H3K27me3-, H3K9me3-, H3K9ac- and H3K4me1-modified nucleosomes, and higher ratio of H3K9ac to H3K4me1 (FIG. 2d). Interestingly, the combinatorial pattern of H3K9me3+H3K36me3-modified nucleosomes decreased in CRC, concomitant with an increase in ‘bivalent’ nucleosomes marked by H3K4me3+H3K27me3 (FIG. 2d). As bivalent chromatin is strongly implicated in many types of cancers^{21,34}, this result further confirms the diagnostic value of single-molecule quantification of combinatorial histone marks. DNA methylation was reduced in CRC samples, in agreement with previous studies⁵⁻³⁶ (FIG. 2E).

The combinatorial pattern of H3K4me1 and H3K9ac is lower in early CRC patients compared to healthy (FIG. 2F). Also, the present teachings can be harnessed towards monitoring patients' positive or negative response to treatment, and the power of collecting multiple layers of information from each sample.

According to a specific embodiment, the disease is colorectal cancer (CRC) and said phenotype is selected from the group consisting of:
(i) increased concentration of nucleosomes as compared to same in a non-cancerous biological fluid;
(ii) increased percentage of modified nucleosome in the biological fluid and wherein said modified nucleosome is comprises H3K27me3-, H3K9me3-, H3K9ac- and H3K4me1;
(iii) higher ratio between H3K9ac to H3K4me1 as compared to same in a non-cancerous biological fluid;
(iv) a decrease in nucleosomes having a bivalent pattern of H3K9me3+H3K36me3- and an increase in nucleosomes having a bivalent pattern of H3K4me3+H3K27me3 as compared to same in a non-cancerous biological fluid.

According to a specific embodiment of this aspect of the invention, the target molecule is a histone modification.

According to a specific embodiment of this aspect of the invention, the histone modification comprises at least one of: H3K9me3, H3K27me3, H3K4me3, H3K36me3, H3K9ac and H3K4me1.

According to a specific embodiment of this aspect of the invention, the at least one comprises at least two.

According to a specific embodiment of this aspect of the invention, the at least two comprise 2, 3, 4, 5 or 6.

According to some embodiments of the invention, screening of the subject for a specific disease is followed by substantiation of the screen results using gold standard methods. These include, tissue biopsies, protein markers, staining, imaging and the like.

It will be appreciated that examination of protein markers such as by using TIRF microscopy at the single molecule level can also be pursued as exemplified in the Examples section which follows. For example, cancer-associated proteins, e.g., CEA, TIMP1 or MST1, mutant oncoproteins (e.g., RAS, BRAF, PIK3CA, and EGFR), mutant tumor suppressor proteins (e.g., p53, CDKN2A, PTEN, RB, APC, SMAD) or pathogen-encoded oncoproteins (e.g. E6 and E7 from HPV).

The present inventors have configured a sensitive method for single molecule protein in detection in fluid biological samples such as plasma, which negates the need for sample processing or dilution. The assay is based on TIRF microscopy in which the native (unprocessed) sample is contacted with a solid-support-immobilized capture antibody and a soluble detection antibody being directed to distinct epitopes on the same protein target, so as to form a “sandwich” by negating the need to wash unbound detection antibody and imaging the sample in real time using time lapse imaging, the signal is accumulated over the period of imaging in which association and dissociation events are monitored thereby increasing assay sensitivity. When immobilizing a number of capture molecules to different proteins and reacting the support with the relevant detection antibodies, multiplex data can be obtained. Harnessing this assay to clinical diagnostic applications ensures sensitive (early) non-invasive disease detection such as solid tumors and may be used in selecting relevant treatments.

Thus according to an aspect of the invention there is provided a method of detecting at least one protein of interest, the method comprising:
(a) contacting a liquid biological sample with a solid support having immobilized thereto at least one capture antibody to said at least one protein of interest, wherein said contacting is under conditions which allow formation of immunocomplexes; and
(b) contacting said immunocomplexes with at least one labeled detection antibody to said at least one protein of interest, wherein said contacting is under conditions which allow formation of immunocomplexes between said immunocomplexes and said labeled detection antibody;
(c) imaging in a time-lapse manner said solid support using Total Internal Reflection (TIRF) microscopy such that said at least one protein of interest in said immunocomplexes is visualized via said labeled antibody, wherein said imaging is under non-flow conditions, thereby detecting the at least one protein of interest.

The present teachings integrate the capabilities of TIRF microscopy, real-time imaging (by time lapse imaging) with the configuration of a sandwich immunoassay in which there is a detection antibody in the liquid phase and a capture antibody bound to the solid support. The detection antibody may be directly or indirectly labeled such as with a secondary antibody. The detection antibody may be added to the plasma prior to contacting with the solid support or after.

The antibodies may be intact antibodies or antibody fragments capable of binding the target protein (different epitopes thereon to avoid competition). They can be monoclonal or polyclonal antibodies. According to a specific embodiment at least one capture antibody is a polyclonal antibody. According to a specific embodiment, the capture antibody is a polyclonal antibody and the detection antibody is a monoclonal antibody.

According to a specific embodiment, the at least one capture antibody comprises a plurality of capture antibodies to distinct proteins of interest and wherein the at least one labeled detection antibody comprises a plurality of labeled detection antibodies to said comprising a plurality of distinct labels. As used herein “plurality” refers to at least 2. According to a specific embodiment, plurality can be 3-30, e.g., 3-25, 3-20, 3-15, 3-10, 3-5.

Methods of immobilizing antibodies to a solid support are well known in the art.

For example, the capture antibody maybe conjugated to the solid support by a molecule having: (a) one or more reactive groups selected from the group comprising: succinimidyl valerate, N-hydroxysuccinimide ester, imidoester, epoxide, isothiocyanate, isocyanate, sulfonyl chloride, aldehyde, carbodiimide, acyl azide, anhydride, fluorobenzene, carbonate, fluorophenyl ester, or a combination thereof; and (b) one or more passivation groups with or without biotin modification, selected from the group comprising polyethylene glycol, polyacrylamide, poly(acrylic acid), poly(N-hydroxyethyl acrylamide), poly(2-hydroxyethyl methacrylate), poly(2-methacryloyloxyethyl phosphorylcholine), poly(vinyl alcohol), poly(vinyl pyrrolidone), hydroxyethylcellulose, hydroxypropyl methylcellulose, dextran, hyaluronic acid, or a combination thereof. Alternatively, the immobilization can rely on the use of a binding pair having affinity between the pair such as avidin-biotin. According to a specific the solid support, e.g., glass, is PEG-avidin-coated solid support.

According to a specific embodiment, the capture antibody is biotinylated.

As mentioned, suitable samples used in the embodiments of the present invention include whole blood, plasma, serum, RBC fraction, urine, saliva, cerebrospinal fluid, semen, sweat, bile, gastric contents, breast milk, exudates, ascites, lymph, sputum, lavage fluid, and bronchial fluid. The sample is a preferable a human sample.

As mentioned hereinabove, the liquid biological sample comprises, in a preferred embodiment, plasma.

According to a specific embodiment, the biological fluid sample is undiluted and/or unprocessed.

Examples of a protein of interest is a tumor-specific nucleocytoplasmic protein; a mutant oncoprotein or a combination thereof; a mutant tumor suppressor protein or a combination thereof; and a pathogen-encoded an oncoprotein derived from an oncogenic pathogen or a combination thereof as examples.

According to a specific embodiment, the at least one protein of interest is a non-secreted tumor specific plasma protein.

According to a specific embodiment, the at least one protein of interest is a secreted tumor specific plasma protein.

Examples of labels which can be used according to the present teachings are well known in the art and exemplified herein in the document.

According to a specific embodiment, the labeled detection antibody comprises a fluorophore.

According to a specific embodiment, the at least one protein of interest is selected from the group consisting of a mutant oncoprotein, a mutant tumor suppressor protein and a pathogen-encoded oncoprotein derived from an oncogenic pathogen.

According to a specific embodiment, the at least one protein of interest is selected from the group consisting of p53, MST1, CEA, and TIMP-1.

According to a specific embodiment, the at least one protein of interest is selected from the group consisting of p53, TIMP-1, MST1, CEA, RAS, KRAS, BRAF, PIK3CA, EGFR, NOTCH1, P53, CDKN2A, PTEN, RB, APC, SMAD, ARID1A, MLL2, MLL3, GATA3, VHL and PBRM1.

According to a specific embodiment, for protein analysis PEGylated-Biotin coated coverslips are assembled and coated with streptavidin. Biotinylated antibodies are incubated on surface followed by wash typically with the same buffer which is compatible with immune complexation i.e., antibody-antigen binding (e.g., IMB2). Next, plasma sample is added and incubated on the surface, typically followed by washing in a buffer which allows and maintains binding of target proteins. Fluorescently labeled antibodies (detection antibodies) are introduced to the surface.

According to a specific embodiment, no washes take place before imaging, i.e., of the detection antibody. This way imaging is effected in the presence of unbound labeled detection antibody to monitor association-dissociation events between said labeled detection antibody and said protein of interest.

Thus, according to a specific embodiment, the imaging is performed without prior washing of said at least one labeled detection antibody. This is exemplified in the Examples section on the target protein p53.

According to a specific embodiment, the biological fluid sample is plasma and a volume of said plasma is less than 1 ml.

TIRF microscopy is already described above and here too, imaging is performed in a time-lapse manner e.g., for 1-24 hours.

This biomarker analysis can be harnessed for diagnosis and optionally treatment of relevant diseases. Thus, according to an aspect of the invention there is provided a method of diagnosing and optionally treating a disease associated with a protein of interest, the method comprising detecting the protein in a biological fluid sample of a subject in need thereof as described herein, wherein presence or level of said protein is indicative of the disease.

Alternatively or additionally, there is provided a method of treating a subject diagnosed with a disease associated with modified, cell-free nucleosomes (cfNucleosomes) in a subject, the method comprising:
(a) affirming diagnosis of the disease according to the teachings disclosed herein;
(b) administering a treatment to the disease.

Alternatively or additionally there is provided use of a medication (e.g., anti cancer treatment) for treating a subject selected according to the diagnosis methods described herein.

Alternatively or additionally, the present teachings can be used for monitoring treatment since a change in histone modifications before and after treatment correlates with disease stage.

As shown in the Examples section which follows, cancer patients were sampled a number of times before and after treatment e.g., tumor resection and extensive treatments. The results of TIRF microscopy according to the present teachings show that the method is sensitive enough to monitor treatment, i.e., positive or negative response to treatment.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Cell-free nucleosomes in mammalian (e.g., human) plasma provides access to molecular information about the pathological processes in the organs or tumors from which it originates. These nucleosomes are derived from fragmented chromatin in dying cells, and retain some of the cell of origin histone modifications.

Hence the present teachings can be used to identify the tissue origin of the cfNucleotosmes and hence can be used to identify the tissue origin of the disease, e.g., malignancy.

Thus, according to an aspect of the invention there is provided a method of identifying a tissue origin of a nucleosome molecule, the method comprising analyzing nucleosome according to the method as described herein, wherein abundance or pattern of the target molecule on the nucleosome is indicative of the tissue origin of the nucleosome.

As used herein “abundance” relates to the number of the modification at the single molecule level.

As used herein “pattern” relates to the position or in combination with other target molecules at the single molecule level.

More specifically, the data obtained can be compared to publicly available data e.g., databases which comprise cfChIP-Seq data which already maps the modification patterns characterizing human tissues, to arrive at the tissue origin of the cfNucleosomes (see also Sadeh et al. Nat Biotechnol. 2021 May 1; 39(5): 586-598).

Thus, the present teachings provide robust and multiplex data at the single molecule level or lower, of nucleosomes, protein markers and tissue origin of nucleosomes. All this using non-invasive methods which will assist in treatment of subject in need thereof.

In another aspect, the present invention provides for a kit comprising: a solid support, a template-dependent DNA polymerase such as Klenow fragment of any other polymerase described herein, a TdT and dATP a portion of which may be labeled. The kit may also include at least one of imaging buffer; labeling ligand(s) such as antibodies for detecting the target molecules and optionally instructions.

In another aspect, the present invention provides for a method of screening chemical compounds that modulate chromatin comprising: incubating the nucleosomes with a chemical compound; and optionally incubating another population (or aliquot) of nucleosomes of the same origin with a control vehicle; and analyzing the nucleosomes prior to and following the incubation or win present or absence of the chemical compounds, wherein a change in composition of the nucleosomes is indicative that the compound modulate.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Patients

All clinical studies were approved by the Israeli Ministry of Health ethics committee (Helsinki applications 091-2020 and 0198-14-HMO). Informed consent was obtained from all individuals before blood sampling.

### Plasma Collection

Blood samples were collected in VACUETTE K3 EDTA tubes and transferred immediately to ice. Next, blood was centrifuged (10 minutes, 1,500 g, 4° C.) and the supernatant was transferred to a fresh 50-ml tubes and centrifuged again (10 minutes, 3,000 g, 4° C.). The supernatant was collected and used as plasma for all experiments. Plasma was analyzed fresh or flash-frozen and stored at −80° C. for future analysis.

Cell-Free Nucleosomes (cfNucleosomes) Preparation for Single-Molecule Imaging

Tagging and tailing of cfNucleosomes was carried out as following: 20 μl of plasma or 5× (DDW diluted) concentrated apoptotic medium was incubated at 37° C. for 1 hour with the following reaction mixture: 10 μl 10× Green Buffer (Enzymatics, B0120), 416 μM CoCl2 (Enzymatics, B0220), 1:60 PI (SIGMA, P8340), 83.3 nM fluorescently labeled dATP (Jena Bioscience, NU-1611-Cy3/Cy5), 83.3 uM dATP (Thermo Fisher Scientific, R0181), 5 μl of Klenow Fragment (3′→5′ exo-, NEB, M0212S) 3 μl of T4 Polynucleotide Kinase (NEB, M0201L) and 4 μl of Terminal deoxynucleotidyl transferase (TdT, Enzymatics, P7070L). Following incubation, samples were inactivated by immediate transfer to ice. For nucleosome sequencing, 1.67 μl of ddATP was added (SIGMA, GE27-2051-01).

Plasma Cell-Free DNA (cfDNA) Isolation and Fluorescent Labeling

cfDNA was extracted from 4 ml of healthy human blood plasma, or from 0.5 ml of plasma from CRC patients, using the Mag-Bind cfDNA Kit (Omega Bio-Tek, M3298-01). For optimized yield, protocol was modified by increasing elution time to 20 minutes on a thermomixer, at 1,600 rpm, in 15 μl elution buffer at room temperature. Sample concentration was measured using Qubit Fluorometer (Thermo Fisher Scientific). For fluorescent labeling of plasma isolated DNA, 10 μl of cfDNA was incubated at 37° C. for 1 hour with the following reaction mixture: NEBuffer™ 2 (NEB, B7202), 0.25 mM MnCl2 (SIGMA, M1787), 33 μM fluorescently labeled dATP (Jena Bioscience, NU-1611-Cy3), 1.5 μl of Klenow Fragment (3′→5′ exo-, NEB, M0212S) and 1.5 μl of T4 Polynucleotide Kinase (NEB, M0201L). Following incubation, samples were inactivated by addition of EDTA (Invitrogen, 15575-038) at a final concentration of 20 mM. Next, DNA was purified by AMPure SPRI beads (Beckman Coulter, A63881), and quantified by Qubit (Thermo Fisher Scientific).

### Cell Culture and Apoptosis

Cell lines were maintained at 37° C. with 5% CO2. HEK-293 cells were cultured in 150 cm plates (10×10⁶ cells in 20 ml of media) in DMEM supplemented with 10% FBS and 1% P/S, and passaged every week. For induction of apoptosis, 6 μM of Staurosporine (STS, Holland-Moran, 62996-74-1.25) was added to medium of confluent cells. 72 hours later, medium was collected and immediately processed. To verify fragment sizes along with nucleosome labeling, 10 ul of the nucleosomes and 10 ul of AMPure extracted DNA (either directly from concentrated medium or after the tagging and tailing reaction), were loaded on High Sensitivity D1000 ScreenTapes (Agilent, 5067-5584) and 6% TBE gel (ThermoFisher Scientific, EC62655BOX), and imaged with 4200 TapeStation (Agilent) or Typhoon imager (Amersham Biosciences), respectively. Apoptotic medium cfNucleosomes were concentrated and recovered using Centricon Plus-70 centrifugation filteres (Merck, UFC710008) according to the manufacture protocol. PI was supplemented 1:100 following concentration.

### Surface Preparation for Single-Molecule Imaging

PEGylated-Biotin and PEGylated-poly T coated microscope slides were prepared based on the protocol described by Chandradoss et al⁴³. Ibidi glass coverslips (25 mm×75 mm, IBIDI, TBD-10812) were cleaned with (1) MilliQ H2O (3× washes, 5 minutes sonication, 3× washes); (2) 2% Alconox (SIGMA, 242985) (20 min sonication followed by 5× washes with MilliQ H2O); and (3) 100% Acetone (20 min sonication followed by 3× washes with MilliQ H2O). Slides were further cleaned and functionalized (Hydroxylated) by incubation in 1 M KOH (SIGMA, 484016) solution for 20 minutes while sonicated, followed by 3× washes with MilliQ H2O. Slides were sonicated twice for 10 minutes in 100% HPLC ethanol (J. T baker 8462-25) prior to applying amino-silanization chemistry. Next, slides were incubated for 24 minutes in a mixture of 3% 3-Aminopropyltriethoxysilane (ACROS Organics, 430941000) and 5% acetic acid in HPLC EtOH, with 1 minute sonication in the middle. Slides were then washed with HPLC EtOH (3×) and MilliQ H2O (3×) and dried with N2. Surface functionalization along with first passivation step was performed by applying mPEG: PEGylated-Biotin/PEG-Azide solution [20 mg PEGylated-Biotin (Laysan, Biotin-PEG-SVA-5000), 180 mg mPEG (Laysan, MPEG-SVA-5000) or 20 mg PEG-Azide (JenKem, A5088), 180 mg mPEG (Laysan, MPEG-SVA-5000)] dissolved in 1560 ul 0.1 M Sodium Bicarbonate (SIGMA, S6297) and degassed (centrifugation at 1 minute at 16,000 g). Next, 140 μl of solution was applied on one surface, followed by immediate assembly of another surface on top. Each pair of assembled surfaces were incubated overnight in a dark humid environment.

For PEGylated-Biotin surfaces: At the next day, surfaces were washed with MilliQ H2O and dried with N2 followed by a second passivation step. MS(PEG)4 (ThermoFisher Scientific, TS-22341) was diluted in 0.1 M of sodium bicarbonate to a final concentration of 11.7 mg/ml and applied on one surface, followed by the assembly of another surface on top. Each pair of assembled surfaces were incubated overnight in dark humid environment. The next day, surfaces were disassembled, washed with MilliQ H2O and dried with nitrogen. After nitrogen flush, surfaces were stored in −20° C.

For PEGylated-poly T surfaces, following PEG-Azide coating, surfaces were washed with MilliQ H2O and dried with N2. To enable anchoring of dT50 to surface via click chemistry, 10 μM of 5′heyxynyl-dT50 (IDT) were mixed with 2 mM of CuSO4 (SIGMA, C1297) and DDW. Next, 100 μl of the mixture was applied on one surface, followed by immediate assembly of another surface on top. Each pair of assembled surfaces was incubated overnight in a dark humid environment. In the next day, a second passivation step [MS(PEG)4] was carried out, similarly to PEGylated-Biotin preparation. Surfaces were stored in −20° C. post nitrogen flush in a similar fashion.

### Antibody Labeling

Capture and detection antibodies were labeled using Biotin conjugation kit (Abeam, ab201796) and Alexa flour antibody labeling kits (Thermo Fisher Scientific, A20181/A10237/A20186) according to the manufacture protocol. Labeled antibodies were purified by size exclusion chromatography using Bio-Spin 6 columns (Bio-Rad, 7326200) followed by measurement of protein concertation using Nanodrop 2000 at 260 nm.

TIMP-1 Imaging and siRNA Transfections

siRNA transfection was performed using INTERFERin (Polyplus, 409-10) according to the manufacturer's protocol. Briefly, cells were plated in 6-well plates (1.5×10′ in 2.5 ml per well) overnight, and the 200 μl of transfection complex was added directly to medium, at final concentration of 25 nM of siRNA. RNA and protein samples were isolated from cells 72 hours after transfection. The following siRNA was used: SMARTpool: ON-TARGETplus Human TIMP1 siRNA (L-011792-00-0005, Dharmacon). For single-molecule imaging, medium was collected from plates, followed by centrifugation at max speed in 4° C. and collection of supernatant to separate proteins from cell debris. Protein concentration was determined by Pierce™ BCA Protein Assay (Thermo Fisher Scientific, 23225), followed by addition (1:100) of protease inhibitor cocktail (PI, SIGMA, P8340).

### Synthetic DNA Preparation for DNA Methylation Assay

DNA fragments were generated by conventional PCR (primer sites underlined) supplementing the reaction with either methylated (NEB, N0356S) or un-methylated cytosine (Thermo Fisher Scientific, R0181), followed by purification with AMPure SPRI beads. The size (˜200 bp) was chosen to mimic the size of mono-nucleosomal DNA fragments previously identified in blood plasma⁴⁴. Fragment labeling, purification and quantification was performed as described for plasma cfDNA.

Sequence:
(SEQ ID NO: 1)
CATCAATGTATCTTATCATGTCTGTATACCGTCGACCTCTAGCTAGAGCT
TGGCGTAATCATGGTCATAGCTGTTTCCTGTGTGAAATTGTTATCCGCTC
ACAATTCCACACAACATACGAGCCGGAAGCATAAAGTGTAAAGCCTGGGG
TGCCTAATGAGTGAGCTAACTCACA.

### 5-Aza-2-Deoxycytidine Treatment

HEK-293 cells were plated in 150 cm plates (10×10⁶ cells in 20 ml of media) overnight, then treated with 1 uM of 5-Aza-2-deoxycytidine (5-Aza, SIGMA, A3656) or PBS for 4 days. Next, 5×10⁶ cells were collected and washed with PBS supplemented with PT (1:100), followed by centrifugation at 3000 rpm for 3 minutes. Cell pellet was resuspended with 1 ml of 0.05% Igepal (SIGMA, 18896) diluted in PBS (supplemented with PI as mentioned above) and centrifuged again at 3000 rpm for 3 minutes. Next, the pellet was resuspended in Lysis buffer [100 mM Tris-HCl pH 7.5 (Gibco, 115567-027), 300 mM NaCl (J. T Baker, 7647145), 2% Triton® X-100 (SIGMA, 9002 93-1), 0.2% sodium deoxycholate (SIGMA, D6750), 10 mM CaCl₂) (SIGMA, 21115)] supplemented with PI and Micrococcal Nuclease (ThermoFisher Scientific, 88216). The reaction mixture was incubated at 37° C. for 10 minutes and then inactivated by addition of EGTA at a final concentration of 20 mM. Then, lysate was centrifuged for 10 minutes at max speed and supernatant was transferred to a new tube. DNA extraction, fluorescent labeling and quantification was performed as described for plasma cfDNA.

### Single-Molecule Imaging

PEGylated-Biotin and PEGylated-poly T coated coverslips were assembled into an Ibidi flowcell (Sticky Slide VI hydrophobic, IBIDI, IBD-80608) generating a six lane flowcell, which enables imaging of six different samples or various combinations of antibodies on a single surface. For PEGylated-Biotin flowcells, Streptavidin (SIGMA, S4762) was added to a final concentration of 0.2 mg/ml followed by 10 minutes incubation and washing with imaging buffer [IMB: 12 mM HEPES pH 8 (Thermo Fisher Scientific, 15630056), 40 mM TRIS pH 7.5 (Gibco, 115567-027) 60 mM KCL (SIGMA, 60142), 0.32 mM EDTA (Invitrogen, 15575-038), 3 mM MgCl2 (SIGMA, 63069), 10% glycerol (Bio-Lab, 56815), 0.1 mg/ml BSA (SIGMA, A7906) and 0.02% Igepal (SIGMA, I8896)]. For time-lapse imaging experiments (Histone PTMs, p53), prior to sample application, TetraSpeck beads (ThermoFisher Scientific, T7279) diluted in PBS were added and incubated on surface for at least 10 minutes to allow correction for stage drift in image analysis. Imaging was performed on a total internal reflection (TIRF) microscope, Nikon (Ti2 LU-N4 TIRF).

### Histone PTMs Analysis

PEGylated-poly T coated coverslips were assembled as described and further passivated with 5% BSA (Merck, A7906) for 30 minutes followed by wash with IMB. Next, plasma sample containing tailed and fluorescently labeled cfNucleosomes was incubated with antibodies (diluted 1:60) for 30 minutes at room temperature (RT), to allow formation of antibody-cfNucleosomes complexes. Next, samples were loaded on the surface and incubated for 15 minutes to allow hybridization. Flowcell was washed (×3) with IMB, followed by time lapse imaging every 15 minutes, with the three laser channels, across all positions (50 Fields of View (FOVs, 148 μm²) per experiment).

### Protein Biomarkers Analysis

PEGylated-Biotin coated coverslips were assembled and coated with streptavidin. Biotinylated antibodies were incubated on surface in IMB2 [10 mM MES pH 6.5 (Boston Bioproducts Inc, NC9904354), 60 mM KCL, 0.32 mM EDTA, 3 mM MgCl2, 10% glycerol, 0.1 mg/ml BSA and 0.02% Igepal] for 30 minutes, followed by wash with IMB2. Next, plasma sample was added to flowcell and incubated on surface for 30 minutes, followed by washes (3×) with IMB2, to allow binding of target proteins. Fluorescently labeled antibodies (detection antibodies) were introduced to the surface for 60 minutes, washed with IMB2, and imaged.

In the same manner for p53 detection (mutant or Wild Type) the same teachings were applied. Only when fluorescently labeled antibodies (detection antibodies)—Fluorescently labeled antibodies [detection antibodies—both total (AF647) and mutant p53 (AF555) antibodies] were introduced to the surface for 10 minutes followed by time lapse imaging of 10 cycles every 15 minutes, across all positions (50 Fields of View (FOVs, 148 μm²) per experiment), no washes took place before imaging.

### Global DNA Methylation Analysis

PEGylated-Biotin coated coverslips were assembled and coated with streptavidin. 2 μl of MBD2-Biotin (Thermo Fisher Scientific, A11148) was incubated with 8 μl of Cy3 labeled cfDNA fragments for 30 minutes, to allow MBD2-Biotin binding to methylated DNA. Next, the reaction mixture was immobilized on the surface and incubated for 10 minutes, followed by TIRF imaging.

### DNA Hydroxymethylation Analysis

cfDNA was incubated in 25 μl reaction mixture containing 50 mM HEPES buffer (pH 8), 25 mM MgCl₂, 60 μM UDP-6-N₃-Glc (Jena Bioscience, CLK-076Motif) and 12.5 U T4 beta-glucosyltransferase (Thermo Fisher Scientific, E00831) for 2 hours at 37° C. Next, 5 μl DBCO-S-S-biotin (Click Chemistry Tools, 10 mM stock in DMSO) was directly added to the reaction mixture and incubated overnight at 37° C. DNA was cleaned using Oligo Clean & Concentrator (Zymo, D4060), and immobilized on a PEGylated-Biotin streptavidin coated surface, followed by imaging.

### Single-Molecule DNA Sequencing

For single-molecule DNA sequencing of cfNucleosomes, PEGylated-poly T surface was blocked with BSA as described above. Poly-A tailed FluoSpheres (described below) along with TetraSpeck beads and cfNucleosomes were applied to surface. PTMs of plasma cfNucleosomes were imaged over time for 169 FOV, as described above. Then flowcell was washed with Wash A buffer [150 mM HEPES (KOH, pH 7.0), 1×SSC, 0.1% SDS] and Wash B buffer [150 mM HEPES (KOH, pH 7.0), 150 mM NaCl] to evict histones and antibodies, and temperature was increased to 37° C. Sequencing was performed as described previously, using Helicos True Single Molecule Sequencing (www(dot)seqll(dot)com/)^{21,45}. FluoSpheres preparation: FluoSpheres (Carboxylate-Modified Microspheres, Thermo Fisher Scientific, F8789) were conjugated to dA50-amine (IDT), tailed as previously described, and hybridized to the surface to serve as reference points for stage drift correction during alignment of sequencing images.

### Single-Molecule Hydroxymethylation Sequencing

2.5 ng of plasma cfDNA was added to a 25 μl solution containing 50 mM HEPES buffer (pH 8), 25 mM MgCl₂, 60 μM UDP-6-N₃-Glc (Jena Bioscience, CLK-076) and 12.5 U T4 beta-glucosyltransferase (Thermo Fisher Scientific, E00831), and incubated for 2 hours at 37° C. Next, 5 μl DBCO-S-S-biotin (Click Chemistry Tools, 10 mM stock in DMSO) was directly added to the reaction mixture and incubated overnight at 37° C. For 5hmC DNA pulldown, samples were incubated at RT with 15 μl of Streptavidin beads (Thermo Fisher Scientific, Dy-11205D) for 1 hour, followed by 3 washes with 1× wash buffer, and elution in 20 μl of 125 mM TCEP (Thermo Fisher Scientific, TS-77720). 5hmC eluted DNA was poly-adenylated and sequenced on a PEGylated-poly T surface as described above. Metagene profile was generated using ngs.plot.

### Image Analysis

Image analysis was performed with the open-source software Cell Profiler (www(dot)cellprofiler(dot)org/). Image analysis pipelines are available upon request. Briefly, time-lapse images of antibody binding events and TetraSpeck beads are aligned, stacked and summed to one image. Antibody spots can be differentiated from TetraSpeck beads spots based on spot size and intensity. Summed antibodies images are aligned with cfNucleosomes images to count colocalization events.

### Predictive Power Score (PPS)

PPS analysis on the data was conducted using a previously published algorithm (www(dot)github(dot)com/80801abs/ppscore). Briefly, by calculating a cross-validated decisions tree for the target variable (e.g., diagnosis) using only one of the markers, it is possible to determine which of the markers in the datasets contributes most to the target variable. The PPS is normalized to the most common assignment in order to provide a baseline for comparison. Using the PPS rather than a simple correlation measure allows us to account for non-linear effects and provides an alternative formulation for correlation which also treats categorical variables (e.g., diagnosis, or disease state).

### Machine Learning Model for Sample Classification

For sample binary classification, various machine-learning algorithms were trained on the features that showed significant differences between healthy and CRC (FIGS. 3c,d,e, FIGS. 5b,c), and evaluated for their performance using a four-fold cross-validation across all samples. The best predictive performance was achieved by a Logistic Regression classifier. To improve classifier performance, the present inventors conducted additional feature selection by training the classifier on all possible feature combinations out of the significant features aforementioned. Evaluating the resulting Area Under the Curve (AUC) values of repeated (500 iterations) four-fold cross-validation for each combination revealed an optimal cumulative performance of a five feature combination: H3K27me3/Nuc, H3K9me3/Nuc, CEA/MST1, H3K27me3+Nuc (% of cfNucleosomes harboring H3K27me3) and global DNA methylation. To evaluate the classifier overall performance using the selected features, the present inventors performed repeated (10K) 4-fold cross-validation across all samples. For each iteration the sensitivity, specificity, accuracy, precision and the AUC value were calculated and averaged over all iterations. R caret and Rweka packages were used for machine-learning modeling.

### Tissue and Plasma Signatures

The present inventors downloaded and combined two independent Homo sapiens based ChIP-seq tracks for each tissue from the Encyclopedia of DNA Elements (ENCODE,). To generate a unique antibody peak profile for a given tissue, the present inventors discarded reads found to overlap with at least one of the other seven tissues tested, retaining only tissue specific peaks. Of note, brain H3K36me3 peaks were available only for embryonic tissue, therefore were replaced with spleen tissue H3K36me3 peaks. To generate unique plasma H3K4me3 ChIP-seq peaks, the present inventors obtained data from healthy (H) and CRC (C) plasma (n=3 for each) produced by Sadeh et. al.¹³. For each group, reads were intersected and only shared reads across all samples were kept for further analysis. Non-overlapping reads between the overlapping healthy and overlapping CRC reads were defined as the unique plasma signature.

Bootstrapping simulation to analyze single-molecule reads overlap with various tissues Overlap significance was assessed as following: First, single-molecule sequenced plasma antibody aligned reads were extended by 100 bp from each side to resemble nucleosome length. Then, for each chromosome, the present inventors randomly selected a number of 230 bp-long DNA segments that is equivalent to the number of antibody positive plasma reads for this chromosome. Random reads were intersected with each unique tissue/plasma signature and overlapping events were recorded. These bootstrapping simulations were iterated 10K times for each tissue for a given antibody to generate a distribution of overlap by chance. Finally, single-molecule sequenced plasma antibody reads were intersected with all tissue signatures, and contrasted against the corresponding distribution of random overlap for that tissue to evaluate overlap significance (two tailed z-test or Wilcoxon rank sum test). Signature and overlap analysis was performed using an in-house R script (EPINUC-overlap), where minimal overlap was defined as 1 bp overlap.

### Statistical Analysis

All the statistical analysis was conducted using the statistical programming language R. Multiple comparison (FIG. 5d) was calculated for cfNucleosomes, DNA methylation and all other significant parameters via One-way ANOVA, Pairwise comparisons using Wilcoxon rank sum test with continuity correction and Asymptotic K-Sample Brown-Mood Median Test, respectively.

### Data Availability

Datasets generated and analyzed during this study and BED files of plasma sequenced reads are available upon request.

### Code Availability

R code for performing overlap analysis is available at www(dot)github(dot)com/Vadim-Fed/EPINUC-overlap.

### Antibodies

TABLE 1

| Antibody | Vendor | Catalog |
|---|---|---|
| Anti-TIMP1Rabbit polyclonal (Biotin) | Abcam | ab77848 |
| Anti-TIMP1 Rabbit polyclonal | Abcam | ab77847 |
| MST1 Polyclonal Antibody | Thermo | PA587047 |
| | Fisher | |
| | Scientific | |
| MST1 Monoclonal Antibody | Thermo | MA529824 |
| | Fisher | |
| | Scientific | |
| CEA Mouse Monoclonal | Thermo | MA117766 |
| | Fisher | |
| | Scientific | |
| CEA Mouse Monoclonal | Thermo | MIC0101 |
| | Fisher | |
| | Scientific | |
| p53 Mouse Monoclonal | Abcam | ab28 |
| Mutant p53 | Abcam | ab247264 |
| p53 Rabbit Monoclonal (Alexa Fluor ® 647 | Abcam | ab227655 |
| Conjugate) | | |
| Tri-Methyl-Histone H3 (Lys27) Rabbit mAb | CST | C36B11 |
| (AlexaFluor ®488 Conjugate) | | |
| Acetyl-Histone H3 (Lys9) Rabbit mAb (Alexa | CST | C5B11 |
| Fluor ®647 Conjugate) | | |
| Tri-Methyl-Histone H3 (Lys4) Rabbit mAb (In | CST | C42D8 |
| house Alexa Fluor ®647 Conjugation) | | |
| Tri-Methyl-Histone H3 (Lys9) Rabbit mAb | CST | D4W1U |
| (Alexa Fluor ® 488 Conjugate) | | |
| Mono-Methyl-Histone H3 (Lys4) XP ®Rabbit | CST | D1A9 |
| mAb (Alexa Fluor ® 488 Conjugate) | | |
| Tri-Methyl-Histon H3 (Lys36) XP(R)Rabbit | CST | D5A7 |
| mAb (Alexa Fluor ® 647 Conjugate) | | |

### Example 1

### EPINUC, an Embodiment of the Present Methodology

Colorectal Cancer (CRC) is the third most common cancer worldwide, causing approximately 700,000 deaths every year¹⁹. Early metastatic seeding has been recently demonstrated in CRC²⁰, underlining the necessity to develop better diagnostic tools to improve patient outcome. In this study, the present inventors developed a single-molecule-based liquid biopsy approach, to analyze multiple parameters from less than 1 ml of plasma sample and demonstrated its value for CRC diagnosis. They coined the technology “EPINUC” for Epigenetics of Plasma Isolated Nucleosomes (FIG. 1a). The technology builds on the recent development of a single-molecule system to image combinatorial histone modifications by Total Internal Reflection (TIRF) microscopy²¹. To capture nucleosomes from plasma, the present inventors developed high-efficiency enzymatic reactions to fluorescently tag and polyadenylate nucleosomes (Methods). Tailed, intact nucleosomes were then immobilized on a PEGylated surface via hybridization, and the status of their post-translational modifications (PTMs) was recorded by TIRF imaging with fluorescently tagged antibodies (FIG. 1b). Binding and dissociation of antibodies to target PTMs was imaged over 90 minutes, leveraging the TIRF narrow excitation range (˜100 nm). Integration of binding events assured maximal detection of modified histones (FIG. 1c).

EPINUC relies on direct counting of single-molecules in a population, yielding data amenable to absolute quantification and comparisons between samples. Each antibody was verified for specificity and linearity of binding with a panel of recombinant modified nucleosomes, yielding six antibodies that passed the quality control criteria (not shown). These antibodies target the tri-methylations on histone H3 μlysine 9 (H3K9me3) and lysine 27 (H3K27me3), associated with gene silencing and heterochromatin, as well as antibodies targeting marks associated with active transcription: tri-methylation of histone H3 on lysine 4 (H3K4me3) and lysine 36 (H3K36me3), and acetylation on lysine 9 (H3K9ac). In addition, the panel includes an antibody targeting mono-methylation of histone H3 on lysine 4 (H3K4me1), a mark associated with enhancers^{22,23}.

Nucleosomes from each plasma sample were tagged with Cy3 (green), and imaged with three pairwise combinations of antibodies labeled with AF488 (cyan) or AF647 (red). Thus, multi-parametric data was obtained for six histone PTMs, comprising of the percentage of modified nucleosomes in each sample, the ratio between various histone modifications, and the percentage of nucleosomes that harbor a combinatorial pattern of two modifications (FIGS. 1d,e,f). EPINUC is the only technology that enables counting of multiple histone PTMs, as well as combinatorially-modified nucleosomes, at a single-molecule precision, from low volume plasma sample (<1 ml).

To extend the number of analytes measured beyond histone PTMs, the present inventors exploited the single-molecule system for quantification of protein biomarkers. The present inventors modulated surface chemistry to contain PEG-streptavidin, allowing anchoring of biotin-conjugated antibodies that target plasma proteins. Following incubation with plasma, bound proteins are imaged by fluorescent detection antibodies. Multiplexed simultaneous detection of three biomarkers is achieved through the use of distinct fluorophores (FIG. 2a). The present inventors imaged two proteins known to increase in plasma of CRC patients: Carcinoembryonic antigen (CEA), a canonical biomarker measured routinely by clinicians²⁴, and Tissue inhibitor of metalloproteinase-1 (TIMP-1), a glycoprotein reported to have diagnostic value in screening for CRC²⁵. In addition, the mammalian sterile 20-like kinase 1 (MST1), an inhibitor of cell proliferation that decreases in CRC patients²⁶ was measured (FIG. 2b). Linear detection and specificity were verified using cell-culture systems and knockdown experiments (FIG. 2c,d and FIGS. 4a-d).

Counting of single molecules confers high sensitivity^{27,28}, thus the present inventors explored whether they could also quantify non-secreted tumor-specific plasma proteins that are undetectable by conventional technologies. They focused on the tumor suppressor p53, which is frequently mutated in CRC; p53 mutations lead to its stabilization and accumulation in tumor cells²⁹. p53 was captured from plasma and applied simultaneous detection with two distinct antibodies; an antibody targeting both the wild type and mutant forms of p53, or another antibody specifically targeting the mutant conformation (FIG. 2e). Time-lapse imaging enabled the accumulation of p53 signal, overcoming the transient binding dynamics of the detection antibodies (FIG. 2f and FIG. 4d). Indeed, they observed higher levels of total and mutant p53 in the plasma of CRC patients with confirmed p53 mutations (FIG. 2g), establishing the present system's capabilities in specific detection of mutant proteins that originate directly from tumor cells.

DNA methylation is often deregulated in cancer, and specifically in colorectal cancer^{30,31}. The present inventors therefore aimed to combine the above analysis with quantitative single-molecule detection of DNA methylation levels in plasma. They incubated Methyl-CpG-binding domain protein 2 (MBD2-biotin), which specifically binds to methylated DNA³², with fluorescently labeled plasma cfDNA. Bound complexes were anchored to the surface and imaged (FIG. 2h). Specificity and sensitivity were validated using synthetic methylated/unmethylated DNA, as well as DNA from cells treated with the DNA methyl transferase (DNMT) inhibitor 5-Aza-2′-deoxycytidine (FIG. 2i and not shown). Finally, they verified detection of cfDNA methylation levels from plasma of CRC and healthy subjects (FIG. 2j).

EPINUC was used to generate high-dimensional data, comprising of the three layers of information; histone PTMs, DNA methylation and protein biomarkers, from 33 plasma samples of healthy subjects and 29 samples taken from 23 late stage CRC patients (stages III-IV; six patients were sampled twice at different times during cancer progression and treatment). CRC samples were obtained from patients prior to surgery or from patients that underwent surgical resection procedure and chemotherapy. In accordance with its use in clinical diagnostics²⁴, single-molecule counting of CEA showed higher levels in CRC patients (FIG. 3a and FIG. 5a,b), and a reduction in patients after resection. Interestingly, high CEA levels were also observed in a few healthy individuals, generating a ‘false positive’ signal (for example, sample 19, marked by * in FIG. 5a). Simultaneous probing of MST1, an anti-proliferative factor, allowed us to derive the CEA/MST1 ratio, resulting in better classification of samples and highlighting an advantage of combinatorial biomarker detection (FIG. 3b,c). Of note, plasma from CRC patients following resection exhibited altered CEA/MST1 ratio compared to non-resected patients, showing higher similarity to healthy individuals (FIG. 3a,b). This demonstrates the potential applicability of the present technology to monitor treatment, while underlying the need to collect additional information from each sample to allow correct sample classification.

EPINUC also provides quantitative measurements of the total number of cfNucleosomes, six histone PTMs, their pairwise combinations and ratios per plasma sample (FIGS. 1A-F). In agreement with the literature, CRC patients had higher cfNucleosomes in their plasma compared to healthy controls³³ (FIG. 5c). While most epigenetic parameters did not change, several showed significant differences: CRC patients had higher levels of H3K27me3-, H3K9me3-, H3K9ac- and H3K4me1-modified nucleosomes, and higher ratio of H3K9ac to H3K4me1 (FIG. 3d and FIGS. 5c,d). Interestingly, the combinatorial pattern of H3K9me3+H3K36me3-modified nucleosomes decreased in CRC, concomitant with an increase in ‘bivalent’ nucleosomes marked by H3K4me3+H3K27me3 (FIG. 3d). As bivalent chromatin is strongly implicated in many types of cancers^{21,34}, this result further confirms the diagnostic value of single-molecule quantification of combinatorial histone marks. DNA methylation was reduced in CRC samples, in agreement with previous studies⁵⁻³⁶ (FIG. 3e).

The identification of epigenetic and biomarkers alterations in late stage CRC motivated us to apply EPINUC to eight plasma samples from individuals diagnosed with early stage CRC (stage II). As in the later stage, the levels of DNA methylation, CEA and CEA/MST1 ratio significantly differed in early stage cancer patients versus healthy (FIG. 3f and FIG. 5e). Interestingly, TIMP1, whose levels did not alter between the cohort of healthy and late stage CRC, was elevated at the early stage (FIG. 5e,f). This may reflect the downregulation of TIMP1 following chemotherapy³⁷, rendering it a significant biomarker only for early stage. Of note, plasma from stage II CRC patients also showed elevated levels of H3K27me3- and H3K9me3-modified nucleosomes, as seen in the late stage (FIG. 3f). Interestingly, the present inventors did not observe increased levels of cfNucleosomes in early stage CRC, likely due to the low tumor burden (FIG. 5e). While the levels of H3K9ac- and H3K4me1-modified nucleosomes did not differ significantly from the healthy group, the combinatorial pattern of H3K4me1 and H3K9ac was lower in early CRC patients compared to healthy (FIG. 3f). Finally, the present inventors calculated the predictive score of each parameter alone to discriminate between the healthy and the distinct groups of CRC patients (FIG. 3g, Methods). These results highlight EPINUC's capabilities in providing multiplexed single-molecule measurements of protein biomarkers, epigenetic modifications and their combinations for CRC diagnostics.

To visualize the distribution of samples across the most significant and predictive parameters, they performed Principal Component Analysis (PCA). The PCA showed spatial separation between the groups, with the early stage CRC samples positioned in between the healthy and the late-stage CRC, potentially reflecting a transition stage (FIG. 3h). While samples from healthy individuals formed a tight cluster, the cancer samples showed greater variability, likely due to inherent heterogeneity between tumors. CRC patients who underwent resection also exhibited a high heterogeneity; interestingly, patients who received both primary tumor resection and metastectomy were positioned closer to the healthy group (samples 4118, 4211 and 4050).

A few CRC patients in the cohort were sampled twice along the course of the study, allowing us to examine the projection of samples taken from the same individual in the PCA plot (FIG. 3h, marked in *, #, and +). Sample 4090 was collected two months following sample 4075 from a CRC patient who underwent tumor resection and extensive treatments. Unfortunately, her condition did not improve and she passed away a month later; indeed the later sample projects further from healthy on both principle components. A similar trend can be seen for samples 3488 and 4059, taken 6.5 months apart. These results highlight the potential of EPINUC to monitor patients' positive or negative response to treatment, and the power of collecting multiple layers of information from each sample.

Finally, in order to integrate all measurements and fine-tune the discrimination between healthy and CRC samples, the present inventors employed machine-learning classification (not shown Methods). The best predictive model displayed high diagnostic potential by generating a 0.96 AUC [95% confidence interval (CI) 0.935-0.981], and sensitivity of 88% [95% CI 82.9-93.3] at 90% specificity [95% CI 84-94.8] and 91% precision [95% CI 87.1-95.3], outperforming predictive models relying solely on protein biomarkers or DNA methylation coupled with biomarkers. Intriguingly, this high discrimination power is achieved without including DNA sequencing. This is mainly due to the combination of multiple parameters spanning various cellular pathways into a single assay, and the high accuracy of the single-molecule technology that allows for digital counting of molecules.

It was hypothesized that introducing a sequencing feature for samples that were classified as cancerous by the machine-learning algorithm would provide yet another layer of specificity and sensitivity. As different tissues vary in their epigenetic modifications, it may allow detection of the tissue-of-origin of the circulating nucleosomes, thus revealing the origin of the cancer. To that end, the present inventors coupled the epigenetic analysis with single-molecule DNA sequencing²¹ (not shown). Briefly, following detection of histone PTMs on cfNucleosomes, the histone proteins are evicted, and the DNA is subjected to repeated cycles of sequencing-by-synthesis using an automated fluidics system. Each cycle consists of incorporation of A, C, T or G by DNA polymerase and imaging; following 120 cycles, the data is integrated to build a strand that can be aligned to the genome, corresponding to the position of the modified nucleosome.

As proof-of-concept, the present inventors applied EPINUC followed by sequencing (EPINUC-seq) to two plasma samples of late stage CRC probed for H3K4me3 and H3K27me3 (Methods). Single-molecule mapped reads, corresponding to modified nucleosomes, were intersected with unique antibody peak signatures generated from ENCODE ChIP-seq data for various tissues and primary cell lines, followed by bootstrapping simulations to calculate significance. Reinforcing the hypothesis, the present inventors found that both plasma samples showed significant overlap with colon-specific H3K4me3 and H3K27me3 peaks, indicating colon as the main tissue-of-origin (not shown). Validating the approach, a similar analysis of a plasma sample from lung cancer patient revealed lung tissue as the main origin (not shown). Moreover, comparing the data to a recent ChIP-seq study of H3K4me3 in plasma showed significant overlap with profiles obtained from CRC patients¹³, but not with healthy plasma. H3K27me3 mapped reads showed a broader pattern, overlapping with peaks corresponding to hematopoietic lineage as well as colon. Interestingly, they also observed a significant overlap with liver-specific H3K27me3 peaks, in agreement with clinical analysis indicating this CRC patient (patient 4044) had liver metastasis.

The present work establishes EPINUC as a novel liquid biopsy approach that analyzes multiple histone and DNA modifications, as well as protein biomarkers, at single-molecule precision. EPINUC distinguishes between CRC patients to healthy individuals at high specificity and sensitivity. The present inventors showed that this multi-parametric approach is suitable also for detection of early stage patients, although expanding the analysis to a larger cohort is needed. The main challenges with analyzing plasma nucleosomes are (1) their minute amount—in 1 ml of plasma there are ˜1000 genome copies^{13,42}; (2) The plasma is highly dense with additional proteins, rendering enzymatic or binding approaches to capture nucleosomes difficult; (3) There is high variability between different individuals, stressing the need for quantitative methodologies to allow comparison between samples; and (4) Multi-parametric data is needed to achieve high specificity and confidence in detection. The EPINUC approach addresses these challenges by enabling single-molecule combinatorial detection of epigenetic marks, DNA sequencing and protein biomarkers from limited input material. In addition to the unique epigenetic analysis, the single-molecule system outperforms the classical ELISA assay for measuring protein biomarkers. ELISA is of relatively low sensitivity and is therefore limited to proteins that are present at high levels, has lower dynamic range in quantifying proteins, and is not amenable to multiplexed detection of several proteins^{27,28}. The present inventors showed that the single-molecule system is capable of detecting the mutant form of p53, which is a non-secreted protein that originates directly from the tumor cells. Importantly, the system is straightforward to adapt for detection of additional proteins, thus increasing sensitivity and enabling disease-specific biomarkers analysis.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited in the Document

ADDIN Mendeley Bibliography CSL_BIBLIOGRAPHY 1. Wan, J. C. M. et al. Liquid bioapsies come of age: Towards implementation of circulating tumour DNA. Nat. Rev. Cancer 17, 223-238 (2017).
2. Bronkhorst, A. J., Ungerer, V. & Holdenrieder, S. The emerging role of cell-free DNA as a molecular marker for cancer management. Biomol. Detect. Quantif 17, 100087 (2019).
3. Heitzer, E., Haque, I. S., Roberts, C. E. S. & Speicher, M. R. Current and future perspectives of liquid biopsies in genomics-driven oncology. Nat. Rev. Genet. 20, 71-88 (2019).
4. Lo, Y. M. D., Han, D. S. C., Jiang, P. & Chiu, R. W. K. Epigenetics, fragmentomics, and topology of cell-free DNA in liquid biopsies. Science (80-.). 372, (2021).
5. Xu, R. H. et al. Circulating tumour DNA methylation markers for diagnosis and prognosis of hepatocellular carcinoma. Nat. Mater. 16, 1155-1162 (2017).
6. Moss, J. et al. Comprehensive human cell-type methylation atlas reveals origins of circulating cell-free DNA in health and disease. Nat. Commun. 9, 5068 (2018).
7. Kang, S. et al. CancerLocator: Non-invasive cancer diagnosis and tissue-of-origin prediction using methylation profiles of cell-free DNA. Genome Biol. 18, 1-12 (2017).
8. Shen, S. Y. et al. Sensitive tumour detection and classification using plasma cell-free DNA methylomes. Nature 563, 579-583 (2018).
9. Reinberg, D. & Vales, L. D. Chromatin domains rich in inheritance only certain histone posttranslational modifications qualify as being epigenetic. Science (80-.). 361, 33-34 (2018).
10. Shema, E., Bernstein, B. E. & Buenrostro, J. D. Single-cell and single-molecule epigenomics to uncover genome regulation at unprecedented resolution. Nat. Genet. 51, 19-25 (2019).
11. Allis, C. D. & Jenuwein, T. The molecular hallmarks of epigenetic control. Nat. Rev. Genet. 17, 487-500 (2016).
12. Mancarella, D. & Plass, C. Epigenetic signatures in cancer: proper controls, current challenges and the potential for clinical translation. Genome Med. 2021 131 13, 1-12 (2021).
13. Sadeh, R. et al. ChIP-seq of plasma cell-free nucleosomes identifies gene expression programs of the cells of origin. Nat. Biotechnol. 39, 586-598 (2021).
14. Gezer, U. et al. Histone methylation marks on circulating nucleosomes as novel blood-based biomarker in colorectal cancer. Int. J. Mol. Sci. 16, 29654-29662 (2015).
15. Van den Ackerveken, P. et al. A novel proteomics approach to epigenetic profiling of circulating nucleosomes. Sci. Rep. 11, 1-12 (2021).
16. Snyder, M. W., Kircher, M., Hill, A. J., Daza, R. M. & Shendure, J. Cell-free DNA Comprises an in Vivo Nucleosome Footprint that Informs Its Tissues-Of-Origin. Cell 164, 57-68 (2016).
17. Ulz, P. et al. Inferring expressed genes by whole-genome sequencing of plasma DNA. Nat. Genet. 48, 1273-1278 (2016).
18. Sun, K. et al. Orientation-aware plasma cell-free DNA fragmentation analysis in open chromatin regions informs tissue of origin. Genome Res. 29, 418-427 (2019).
19. Ferlay, J. et al. Cancer incidence and mortality worldwide: Sources, methods and major patterns in GLOBOCAN 2012. Int. J. Cancer 136, E359-E386 (2015).
20. Hu, Z. et al. Quantitative evidence for early metastatic seeding in colorectal cancer. Nat. Genet. 51, 1113-1122 (2019).
21. Shema, E. et al. Single-molecule decoding of combinatorially modified nucleosomes. Science (80-.). 352, 717-721 (2016).
22. Heintzman, N. D. et al. Distinct and predictive chromatin signatures of transcriptional promoters and enhancers in the human genome. Nat. Genet. 39, 311-318 (2007).
23. Barski, A. et al. High-Resolution Profiling of Histone Methylations in the Human Genome. Cell 129, 823-837 (2007).
24. Tiernan, J. P. et al. Carcinoembryonic antigen is the preferred biomarker for in vivo colorectal cancer targeting. Br. J. Cancer 108, 662-667 (2013).
25. Meng, C. et al. TIMP-1 is a novel serum biomarker for the diagnosis of colorectal cancer: A meta-analysis. PLoS One 13, e0207039 (2018).
26. Yu, J. et al. Identification of MST1 as a potential early detection biomarker for colorectal cancer through a proteomic approach. Sci. Rep. 7, (2017).
27. Mandal, S. et al. Direct Kinetic Fingerprinting for High-Accuracy Single-Molecule Counting of Diverse Disease Biomarkers. Acc. Chem. Res. (2020). doi:10.1021/acs.accounts.0c00621
28. Furth, N. et al. Unified platform for genetic and serological detection of COVID-19 with single-molecule technology. PLoS One 16, e0255096 (2021).
29. Nakayama, M. & Oshima, M. Mutant p53 in colon cancer. J. Mol. Cell Biol. 11, 267-276 (2019).
30. Jung, G., Hernindez-Illin, E., Moreira, L., Balaguer, F. & Goel, A. Epigenetics of colorectal cancer: biomarker and therapeutic potential. Nat. Rev. Gastroenterol. Hepatol. 17, 111-130 (2020).
31. Dawson, M. A. The cancer epigenome: Concepts, challenges, and therapeutic opportunities. Science (80-.). 355, 1147-1152 (2017).
32. Wood, K. H. & Zhou, Z. Emerging molecular and biological functions of MBD2, a reader of DNA methylation. Front. Genet. 7, (2016).
33. Bettegowda, C. et al. Detection of circulating tumor DNA in early- and late-stage human malignancies. Sci. Transl. Med. 6, 224ra24-224ra24 (2014).
34. Brown, R., Curry, E., Magnani, L., Wilhelm-Benartzi, C. S. & Borley, J. Poised epigenetic states and acquired drug resistance in cancer. Nat. Rev. Cancer 14, 747-753 (2014).
35. Kerachian, M. A. et al. Crosstalk between DNA methylation and gene expression in colorectal cancer, a potential plasma biomarker for tracing this tumor. Sci. Rep. 10, 1-13 (2020).
36. King, W. D. et al. A cross-sectional study of global DNA methylation and risk of colorectal adenoma. BMC Cancer 14, 1-9 (2014).
37. Frederiksen, C. et al. Plasma TIMP-1 μlevels and treatment outcome in patients treated with XELOX for metastatic colorectal cancer. Ann. Oncol. 22, 369-375 (2011).
38. Li, W. et al. 5-Hydroxymethylcytosine signatures in circulating cell-free DNA as diagnostic biomarkers for human cancers. Cell Res. 27, 1243-1257 (2017).
39. Lio, C. W. J., Yuita, H. & Rao, A. Dysregulation of the TET family of epigenetic regulators in lymphoid and myeloid malignancies. Blood 134, 1487-1497 (2019).
40. Zhang, L. et al. Tet-mediated covalent labelling of 5-methylcytosine for its genome-wide detection and sequencing. Nat. Commun. 4, (2013).
41. Song, C. X. et al. 5-Hydroxymethylcytosine signatures in cell-free DNA provide information about tumor types and stages. Cell Res. 27, 1231-1242 (2017).
42. Newman, A. M. et al. Integrated digital error suppression for improved detection of circulating tumor DNA. Nat. Biotechnol. 34, 547-555 (2016).
43. Chandradoss, S. D. et al. Surface passivation for single-molecule protein studies. J. Vis. Exp. (2014). doi:10.3791/50549
44. Fleischhacker, M. & Schmidt, B. Circulating nucleic acids (CNAs) and cancer-A survey. Biochim. Biophys. Acta—Rev. Cancer 1775, 181-232 (2007).
45. Harris, T. D. et al. Single-Molecule DNA Sequencing of a Viral Genome. Science (80-.). 320, 106-109 (2008).

What is claimed is:

1. A method of analyzing nucleosomes, the method comprising:
(a) isolating a plurality of nucleosome molecules from a biological sample;
(b) enzymatically linking adenine nucleotides to free DNA ends of said plurality of nucleosome molecules, wherein at least a portion of said adenine nucleotides comprises a label, such that said plurality of nucleosome molecules become attached to a labeled poly(A) tail;
(c) hybridizing said plurality of nucleosome molecules attached to said labeled poly(A) tail to a solid support coated with poly(T); and
(d) imaging said solid support, whereby said plurality of nucleosome molecules are visualized.

2. The method of claim 1, further comprising:
(e) incubating said solid support with at least one labeling ligand with specific binding affinity for a target molecule of said nucleosome and wherein the labeling ligand includes a marker;
(f) imaging the solid support, whereby said plurality of nucleosome molecules comprising said target molecule are visualized.

3. The method of claim 1, wherein said enzymatically linking comprises using a template-dependent DNA polymerase and a Terminal deoxynucleotidyl transferase (TdT).

4. The method of claim 3, wherein said template-dependent DNA polymerase comprises a Klenow polymerase.

5. The method of claim 1, wherein said biological sample comprises a biological fluid, optionally plasma, optionally said plasma is less than 1 ml.

6. The method of claim 2, further comprising cleaving said label and optionally washing it prior to step (e).

7. The method of claim 2, wherein said labeling ligand comprises an antibody.

8. The method of claim 2, wherein said target molecule is a post translational modification.

9. The method of claim 2, wherein said target molecule is a histone modification and/or a histone variant optionally said histone variant is selected from the group consisting of macroH2A1.1, macroH2A1.2, H2AZ, H2AX, H3.1 and H3.3.

10. The method of claim 2, wherein said target molecule is a nucleotide modification, optionally said nucleotide modification is selected from the group consisting of 5-methyl-(5-mC), 5-hydroxymethyl-(5-hmC), 5-formyl-(5-fC) and 5-carboxy-(5-eaC) cytosine.

11. The method of claim 2, wherein said imaging of step (f) comprises time lapse imaging, and/or said imaging of step (f) and optionally (d) comprises TIRF microscopy.

12. The method of claim 2, further comprising repeating steps (e) and (f) with additional labeling ligand distinctive of said labeling ligand such as in binding a different target molecule of said nucleosome.

13. The method of claim 2, wherein said imaging of step (e) comprises multiplex imaging.

14. The method of claim 1, wherein said plurality of nucleosome molecules comprise cell-free nucleosomes (cfNucleosomes).

15. The method of claim 1, wherein said solid support is coated with poly ethylene glycol (PEG).

16. The method of claim 1, further comprising sequencing DNA of said plurality of nucleosome molecules and optionally wherein said sequencing comprises sequencing by synthesis.

17. A method of diagnosing a disease associated with modified, cell-free nucleosomes (cfNucleosomes) comprising analyzing nucleosome molecules in a biological fluid according to claim 1, wherein presence of a pathological nucleorise phenotype is indicative of a disease associated with modified cfNucleosomes.

18. The method of claim 1, wherein said phenotype is selected from the group consisting of:
(i) increased concentration of nucleosomes as compared to same in a non-cancerous biological fluid;
(ii) altered percentage of modified nucleosome in a specific target molecule in the biological fluid as compared to same in a non-cancerous biological fluid;
(iii) altered ratio between a plurality of said target molecules as compared to same in a non-cancerous biological fluid;
(iv) altered percentage of nucleosomes that comprise a combinatorial pattern of target molecules in a single nucleosome as compared to same in a biological fluid of a non-cancerous biological fluid.

19. A method of treating a subject diagnosed with a disease associated with modified, cell-free nucleosomes (cfNucleosomes) in a subject, the method comprising:
(a) affirming diagnosis of the disease according to the method of claim 17;
(b) administering a treatment to the disease.

20. A method of analyzing a liquid biological sample, the method comprising analyzing nucleosomes and a protein of interest according to the method of claim 1.
